SEQUENCE LISTING (16 sequences)

SEQ ID NO: 1 - protein, 1210 aa, HCV [sequence omitted]

SEQ ID NO: 2 - DNA, 3636 bp, HCV
atggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggttacgaggcacgccgacgtcattcccgtgcgccggcgaggtgatagcaggggtagcctgctttcgccccggcccatttcctacctaaaaggcagcgcggggggtccgctgttgtgccccgcgggacacgccgtgggcctattcagggccgcggtgtgcacccgtggagtggccaaggcggtggactttatccctgtggagaacctagagacaaccatgagatccccggtgttcacggacaactcctctccaccagcagtgccccagagcttccaggtggcccacctgcatgctcccaccggcagtggtaagagcaccaaggtcccggctgcgtacgcagcccagggctacaaggtgttggtgctcaacccctctgttgctgcaacgctgggctttggtgcttacatgtccaaggcccatggggtcgatcctaatatcaggaccggggtgagaacaattaccactggcagccccatcacgtactccacctacggcaagttccttgccgacggcgggtgctcaggaggcgcttatgacataataatttgtgacgagtgccactccacggatgccacatccatcttgggcatcggcactgtccttgaccaagcagagactgcgggggcgagattggttgtgctcgccactgctacccctccgggctccgtcactgtgtcccatcctaacatcgaggaggttgctctgtccaccaccggagagatccctttctacggcaaggctatccccctcgaggtgatcaaggggggaagacatctcatcttctgtcactcaaagaagaagtgcgacgagctcgccgcgaagctggtcgcattgggcatcaatgccgtggcctactaccgcggacttgacgtgtctgtcatcccgaccaacggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcagcgccggggcaggactggcagggggaagccaggcatctacagatttgtggcaccgggggagcgcccctccggcatgttcgactcgtccgtcctctgtgagtgctatgacgcgggctgtgcttggtatgagctcatgcccgccgagactacagttaggctacgagcgtacatgaacaccccggggcttcccgtgtgccaggaccatcttgaattttgggagggcgtctttacgggcctcacccatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgcgcctcacacctcccttacctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataa

SEQ ID NO: 3 - protein, 1210 aa, HCV
MAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTNGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELMPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECASHLPYLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAGRFEQT

SEQ ID NO: 4 - DNA, 3636 bp, HCV
atggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggttacgaggcacgccgacgtcattcccgtgcgccggcgaggtgatagcaggggtagcctgctttcgccccggcccatttcctacctaaaaggctcctcggggggtccgctgttgtgccccgcgggacacgccgtgggcctattcagggccgcggtgtgcacccgtggagtggccaaggcggtggactttatccctgtggagaacctagagacaaccatgagatccccggtgttcacggacaactcctctccaccagcagtgccccagagcttccaggtggcccacctgcatgctcccaccggcagtggtaagagcaccaaggtcccggctgcgtacgcagcccagggctacaaggtgttggtgctcaacccctctgttgctgcaacgctgggctttggtgcttacatgtccaaggcccatggggtcgatcctaatatcaggaccggggtgagaacaattaccactggcagccccatcacgtactccacctacggcaagttccttgccgacggcgggtgctcaggaggcgcttatgacataataatttgtgacgagtgccactccacggatgccacatccatcttgggcatcggcactgtccttgaccaagcagagactgcgggggcgagattggttgtgctcgccactgctacccctccgggctccgtcactgtgtcccatcctaacatcgaggaggttgctctgtccaccaccggagagatccctttctacggcaaggctatccccctcgaggtgatcaaggggggaagacatctcatcttctgtcactcaaagaagaagtgcgacgagctcgccgcgaagctggtcgcattgggcatcaatgccgtggcctactaccgcggacttgacgtgtctgtcatcccgaccaacggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcagcgccggggcaggactggcagggggaagccaggcatctacagatttgtggcaccgggggagcgcccctccggcatgttcgactcgtccgtcctctgtgagtgctatgacgcgggctgtgcttggtatgagctcatgcccgccgagactacagttaggctacgagcgtacatgaacaccccggggcttcccgtgtgccaggaccatcttgaattttgggagggcgtctttacgggcctcacccatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgcgcctcacacctcccttacctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataa

SEQ ID NO: 5 - DNA, 51 bp, Artificial sequence
  Synthetic primer
cagcagcaggtaagggaggtgtgaggcgcactcttccatctcatcgaactc

SEQ ID NO: 6 - DNA, 21 bp, Artificial sequence
  Synthetic primer
tgtctgtcatcccgaccaacg

SEQ ID NO: 7 - DNA, 40 bp, Artificial sequence
  Synthetic primer
agtgagatctgcggccgcttatcatgtctgctcgaagcgg

SEQ ID NO: 8 - DNA, 33 bp, Artificial sequence
  Synthetic primer
tcacacctcccttacctgctgctgctgctgctg

SEQ ID NO: 9 - protein, 10 aa, Artificial sequence
  Synthetic peptide
DEMEECASHL

SEQ ID NO: 10 - protein, 12 aa, Artificial sequence
  Synthetic peptide
DEMEECASHLPY

SEQ ID NO: 11 - DNA, 51 bp, Artificial sequence
  Synthetic primer
cagcagcagcagcaggaggtgtgaggcgcactcttccatctcatcgaactc

SEQ ID NO: 12 - DNA, 36 bp, Artificial sequence
  Synthetic primer
tgcgcctcacacctgctgctgctgctgctgctgggc

SEQ ID NO: 13 - DNA, 18 bp, Artificial sequence
  Synthetic primer
cccaccttggctgtagtc

SEQ ID NO: 14 - DNA, 20 bp, Artificial sequence
  Synthetic primer
ccatgaatcactcccctgtg

SEQ ID NO: 15 - DNA, 19 bp, Artificial sequence
  Synthetic primer
ccggtcgtcctggcaattc

SEQ ID NO: 16 - DNA, 22 bp, Artificial sequence
  Synthetic primer
cctggaggctgcacgacactca

(19) United States
(12) Patent Application Publication
(10) Pub. No.: US 2005/0120398 A1
(43) Pub. Date: Jun. 2, 2005

(54) Animal model for HCV infection

(75) Inventors: Gururaj Kalkeri, Arlington, MA (US); Ann Kwong, Cambridge, MA (US)

Correspondence Address: MARSHALL, GERSTEIN & BORUN LLP, 6300 SEARS TOWER, 233 S. WACKER DRIVE, CHICAGO, IL, 60606, US

(73) Assignee: Vertex Pharmaceuticals Incorporated, Cambridge, MA (US)

(21) Appl. No.: 10/939,958

(22) Filed: Sep. 13, 2004

Related U.S. Application Data
(60) Provisional application No. 60/502,742, filed on Sep. 12, 2003.
(60) Provisional application No. 60/514,739, filed on Oct. 27, 2003.
(60) Provisional application No. 60/526,410, filed on Dec. 1, 2003.
(60) Provisional application No. 60/588,909, filed on Jul. 16, 2004.

(51) Int. Cl. A01K 67/027; C12N 5/06; A61K 38/05; A61K 31/7056; A61K 31/4709

(52) U.S. Cl. 800/9; 435/325; 435/354; 514/18; 514/49; 514/312

(57) ABSTRACT

The present invention relates to a non-transgenic, non-human animal useful as a model for protease activity and for liver damage, including steatosis.

### FIELD OF THE INVENTION

The present invention relates to a non-transgenic, non-human animal useful as a model for protease activity and for liver damage, including steatosis.

### BACKGROUND OF THE INVENTION

Infection by Hepatitis C virus (“HCV”) is a compelling human medical problem. HCV is recognized as the causative agent for most cases of non-A, non-B hepatitis, with an estimated human sero-prevalence of 3% globally [A. Alberti et al., “Natural History of Hepatitis C,” J. Hepatology, 31., (Suppl. 1), pp. 17-24 (1999)]. Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31., (Suppl. 1), pp. 88-91 (1999)].

Upon first exposure to HCV only about 20% of infected individuals develop acute clinical hepatitis while others appear to resolve the infection spontaneously. In almost 70% of instances, however, the virus establishes a chronic infection that persists for decades [S. Iwarson, “The Natural Course of Chronic Hepatitis,” FEMS Microbiology Reviews, 14, pp. 201-204 (1994); D. Lavanchy, “Global Surveillance and Control of Hepatitis C,” J. Viral Hepatitis, 6, pp. 35-47 (1999)]. This usually results in recurrent and progressively worsening liver inflammation, which often leads to more severe disease states such as cirrhosis and hepatocellular carcinoma [M. C. Kew, “Hepatitis C and Hepatocellular Carcinoma”, FEMS Microbiology Reviews, 14, pp. 211-220 (1994); I. Saito et. al., “Hepatitis C Virus Infection is Associated with the Development of Hepatocellular Carcinoma,” Proc. Natl. Acad. Sci. USA, 87, pp. 6547-6549 (1990)].

HCV is an RNA virus belonging to the Flavi viridae family. The virus is classified as a blood borne pathogen and is transmitted mainly through contact with blood borne products. HCV causes chronic hepatitis, fibrosis, and hepatocellular carcinoma in infected humans. The viral genome consists of a positive strand RNA that is 9.6 Kb in length and encodes 3 structural proteins and 7 non structural proteins. Structural proteins core, envelope 1, and envelope 2 are required for viral assembly and packaging.

Non structural proteins ranging from NS2-5 perform various functions that are necessary for viral replication and polyprotein processing.

The NS proteins are derived by proteolytic cleavage of a 3010-3033 amino acid polyprotein [R. Bartenschlager et. al., “Nonstructural Protein 3 of the Hepatitis C Virus Encodes a Serine-Type Proteinase Required for Cleavage at the NS3/4 and NS4/5 Junctions,” J. Virol., 67, pp. 3835-3844 (1993); A. Grakoui et. al., “Characterization of the Hepatitis C Virus-Encoded Serine Proteinase: Determination of Proteinase-Dependent Polyprotein Cleavage Sites,” J. Virol., 67, pp. 2832-2843 (1993); A. Grakoui et. al., “Expression and Identification of Hepatitis C Virus Polyprotein Cleavage Products,” J. Virol., 67, pp. 1385-1395 (1993); L. Tomei et. al., “NS3 is a serine protease required for processing of hepatitis C virus polyprotein”, J. Virol., 67, pp. 4017-4026 (1993)].

The HCV NS protein 3 (NS3) contains a serine protease activity that helps process the majority of the viral enzymes, and is thus considered essential for viral replication and infectivity. The first 181 amino acids of NS3 (residues 1027-1207 of the viral polyprotein) have been shown to contain the serine protease domain of NS3 that processes all four downstream sites of the HCV polyprotein [C. Lin et al., “Hepatitis C Virus NS3 Serine Proteinase: Trans-Cleavage Requirements and Processing Kinetics”, J. Virol., 68, pp. 8147-8157 (1994)].

The HCV NS3 serine protease and its associated cofactor, NS4A, helps process all of the viral enzymes, and is thus considered essential for viral replication.

There are not currently any satisfactory anti-HCV agents or treatments. The only established therapy for HCV disease is pegylated-interferon and ribarivin treatment. However, interferons have significant side effects [M. A. Wlaker et al., “Hepatitis C Virus: An Overview of Current Approaches and Progress,” DDT, 4, pp. 518-29 (1999); D. Moradpour et al., “Current and Evolving Therapies for Hepatitis C,” Eur. J. Gastroenterol. Hepatol., 11, pp. 1199-1202 (1999); H. L. A. Janssen et al. “Suicide Associated with Alfa-Interferon Therapy for Chronic Viral Hepatitis,” J. Hepatol., 21, pp. 241-243 (1994); P. F. Renault et al., “Side Effects of Alpha Interferon,” Seminars in Liver Disease, 9, pp. 273-277. (1989)] and induce long term remission in only a fraction (˜25%) of cases [O. Weiland, “Interferon Therapy in Chronic Hepatitis C Virus Infection”, FEMS Microbiol. Rev., 14, pp. 279-288 (1994)]. Moreover, the prospects for effective anti-HCV vaccines remain uncertain.

Progress in developing HCV protease inhibitors is hampered by the lack of a robust and reproducible animal model. Many animals are not susceptible to HCV infection.

Chimpanzees are the best understood animal model for HCV (Bassett et al., J. Virol. Feburary;73(2):1118-26 1999; Bassett et al., Hepatology. 29(6):1884-92, 1999 Brasky et al. 1998; Bassett et al., J. Virol. 72(4):2589-99, 1998; Bukh, Hepatology, 39(6):1469-75, 2004; Bukh, Apgar et al. J Infect Dis. 178(4):1193-7 1998; Kolykhalov, Mihalik et al. J. Virol. 2000 February;74(4):2046-51, 2000; Bukh, Forns et al. Intervirology, 44(2-3):132-42, 2001). However, there are ethical issues and problems with cost and availability that are associated with testing drugs in chimpanzees. Furthermore, HCV infection of chimpanzees runs a milder course (Walker, Springer Semin Immunopathol. 19(1):85-98 1997) and the disease spectrum is different from human HCV infections. 70% of HCV infections in humans become chronic infections and 30% of the infected patients clear the virus. In contrast, 65-80% of infected chimpanzees clear the virus and 25-30% infections result in acute hepatitis. Moreover the course of the disease of HCV infection in chimpanzees is milder than in humans and chimpanzees don't develop cirrhosis as a result of HCV infection. The cost of infecting chimpanzees with HCV is around $ 60,000 per animal.

Attempts to infect other non human primate models such as lower primates (Bukh, Apgar et al. J Viral Hepat., 8(3):228-31 2001; Korzaya, Lapin et al. Bull Exp Biol Med., 133(2):178-81, 2002) or baboons (Sithebe, Kew et al. J Med Virol., 66(4):468-71 2002) with HCV have met with mixed results. Surrogate animal models such as GB virus infection of tamarins (Garson, Whitby et al. J Med Virol. 52(3):286-8. 1997); (Bukh, Apgar et al. Virology. 30;262(2):470-8 1999; Beames, Chavez et al. J. Virol., 74(24):11764-72 2000; Beames, Chavez et al., ILAR J., 42(2):152-60, 2001; Sbardellati, Scarselli et al. 2001; Lanford, Chavez et al. Virology., 311(1):72-80 2003; Martin, Bodola et al. Proc Natl Acad Sci USA., 100(17):9962-7 2003) have met with variable success.

Transgenic mice harboring some parts of HCV to study HCV induced liver pathogenesis and hepatocellular carcinoma (HCC) have been reported (Koike, Moriya et al. J Gen Virol., 76 (Pt 12):3031-8, 1995; Kawamura, Furusaka et al. 1997; Moriya, Yotsuyanagi et al. J Gen Virol., 78 (Pt 7):1527-31, 1997; Pasquinelli, Shoenberger et al. Hepatology., 25(3):719-27 1997; A. Honda et al., J. Med. Virol., 59, pp. 281-289 (1999); Koike Nippon Rinsho., 59(7):1265-70 2001; He, Cheng et al. World J Gastroenterol. 9(3):474-8 2003). These transgenic models suffer several drawback, including not adequately modeling the viral life cycle (V. Brass et al., Hepatology Elsewhere, H. Jaeschke et al., ed., Hepatology, 35, pp. 722-724 (2002)). Mice with chimeric human livers have also been reported in literature. SCID mice transplanted with HCV infected human PBMCs have been reported to demonstrate HCV persistence for 8 weeks post inoculation, but only 2 out of eight mice showed the presence of the replicative (negative strand) form of HCV which is indicative of viral replication (Bronowicki, Loriot et al. Hepatology., 28(1):211-8, 1998). Nude mice transplanted with HCV bearing tumors resulted in marginal HCV replication (Labonte, Morin et al. J Med Virol., 66(3):312-9, 2002). Trimera mouse model with transplantation of HCV bearing tumors in kidney capsule (Galun, Burakova et al., J Infect Dis., 172(1):25-30, 1995; Ilan, Arazi et al. J Infect Dis.,185(2):153-61 2002; Dagan and Eren, Curr Opin Mol Ther., 5(2): 148-55, 2003) and the successful repopulation of HCV infected human hepatocytes in SCID-uPA mice (D. F. Mercer et al., Nature Medicine, 7, pp. 927-933 (2001)) has also been demonstrated. However this model is technically demanding and there is a lot of variability associated with the model as it depends on the variable repopulation of hepatocytes.

A small animal (e.g., a mouse) model would conserve compound and would allow scientists to study the pharmacokinetics (PK) effects of a compound, which is dependent on absorption, distribution, metabolism and toxicity of the antiviral compound; and study the pharmacodynamic (PD) effects of a compound, namely the in vivo efficacy of the compound. Because the currently available mouse models for HCV, such as mice with human liver repopulation models, are highly variable and not robust, they are unsuitable for anti viral drug screening.

Furthermore, it is unclear whether liver injury is caused directly by HCV infection (N. Fausto, Nature Medicine, 7, pp. 890-891 (2001). A model for HCV-related liver injury would provide insight into the infection process and allow for screening of agents to protect against liver damage.

Steatosis is an accumulation of fat in the liver or other parts of the body. Steatosis has been observed in patients with HCV infection. However, other diseases have steatosis as a symptom. See, A. Lonardo et al. “Steatosis and Hepatitis C Virus: Mechanism and Significance for Hepatic and Extrahepatic Disease” Gastroenterology, 126, pp. 586-597 (2004); M. Romero-Gómez et al. “Serum Leptin Levels Correlate with Hepatic Steatosis in Chronic Hepatitis C” 98, pp. 1135-1141 (2003); V. Ratziu et al. “Fat, Diabetes, and Liver Injury in Chronic Hepatitis C” 6, pp. 22-29 (2004); F. Ramalho “Hepatitis C Virus Infection and Liver Steatosis” 60 pp. 125-127 (2003). These diseases, such as nonalcoholic fatty liver diseases (NAFLD), are fairly widespread. Research into therapies for these diseases is hampered by the lack of an adequate steatosis model.

Therefore, there is a need for a more robust, technically less demanding (and therefore reproducible), inexpensive, small animal model suitable for antiviral drug screening and for use as a liver damage model.

### SUMMARY OF THE INVENTION

The present invention relates to an animal model for protease activity. In particular, the invention provides an animal that has a protease-SEAP reporter construct in its liver.

The present invention also relates to an animal model for liver damage. This model involves an animal that has an expression construct that encodes a protein that causes liver damage inserted into its liver.

The present invention also relates to an animal model for steatosis and related disorders. This model involves an animal that has a expression construct that encodes a protein to be expressed that causes steatosis in its liver.

The present invention also relates to cells from these animals and cells, vectors, and cell lines comprising the gene systems/expression constructs described herein.

The animal models provided by this invention are robust, reproducible, and appropriate for small animals. The models are particularly useful in, e.g., drug discovery and modeling protease activity and liver damage in vivo.

The present invention also provides processes for preparing the animal models and methods for using the models.

### BRIEF DESCRIPTION OF THE FIGURES

FIG. 1 is a schematic diagram of reporter genes expressing wild type (WT) and mutant (MT) HCV protease fused with the secreted alkaline phosphatase (SEAP) reporter.

FIG. 2 (MT HCV NS3•4A SEAP protein) depicts the amino acid sequence of the mutant HCV protease fused to the SEAP protein with 4AB junction of Hepatitis C virus in between (underlined). Amino and carboxy terminal boundaries of non structural protein 3 (NS3) and non structural protein 4A (NS4A) components of HCV protease are marked by the arrows. The serine 139 of NS3 protein (boxed) in the protease active site is mutated to Alanine (Ser->Ala) rendering the protease inactive.

FIG. 3 (WT HCV NS3•4A SEAP protein) depicts the amino acid sequence of the wild type HCV protease fused to the SEAP protein with 4AB junction of Hepatitis C virus in between (shown in underlined). Amino and carboxy terminal boundaries of non structural protein 3 (NS3) and non structural protein 4A (NS4A) components of HCV protease are marked by the arrows.

FIG. 4 (MT HCV NS3•4A SEAP DNA) depicts the nucleotide sequence of the mutant HCV protease cDNA fused to the SEAP open reading frame with 4AB junction of HCV protein in between. Mutations in the active site inactivating the protease but without altering the open reading frame are underlined.

FIG. 5 (WT HCV NS3•4A SEAP DNA) depicts the nucleotide sequence of the wild type HCV protease cDNA fused to the SEAP open reading frame with 4AB junction of HCV protein in between.

FIG. 6 depicts SEAP levels measured as RLU units from the supernatants of mouse hepatocytes transfected with HCV WT/MT NS3•4A SEAP plasmids.

FIG. 7 depicts the effect of a HCV protease inhibitor on SEAP secretion.

FIG. 8 is a schematic diagram of Adenoviral constructs encoding HCV WT/MT protease fused to SEAP reporter gene.

FIG. 9 illustrates SEAP release in the medium in Ad/HCV WT or Ad/HCV MT NS3•4A-SEAP infected mouse hepatocytes.

FIG. 10 depicts the effect of a HCV protease inhibitor on Ad/HCV NS3•4A dependent expression of SEAP in the media of mouse hepatocytes.

FIG. 11 depicts SEAP secretion in the serum of SCID mice infected with Ad/WT and Ad/MT HCV NS3•4A-SEAP.

FIG. 12 depicts the effect of SEAP secretion in the serum of SCID mice infected with Ad/WT and Ad/MT HCV NS3•4A-SEAP upon treatment with a HCV protease inhibitor.

FIG. 13 illustrates morphological differences between a) Ad-WT-HCV NS3•4A-SEAP, b) Ad-WT-HCV NS3•4A-SEAP and treatment with a HCV inhibitor, and c) Ad-MT-HCV NS3•4A-SEAP.

FIG. 14 illustrates a dose response study using a composition of a mixture of D- and L-isomers at the N-propyl-side chain of VX-950 in an animal model of this invention.

FIG. 15 is a summary of the constructs used in this invention and a summary of data obtained.

FIG. 16 is a summary of the activity of an exemplary protease inhibitor in a model according to this invention.

FIG. 17 depicts untreated and treated liver samples from a model according to this invention.

FIG. 18 shows the structure of VX-950 (a mixture of the D- and L-isomers was used) and assay data related thereto.

FIG. 19: Activity of VX-950 (mixture of the D- and L isomers) in the Vertex HCV protease mouse model.

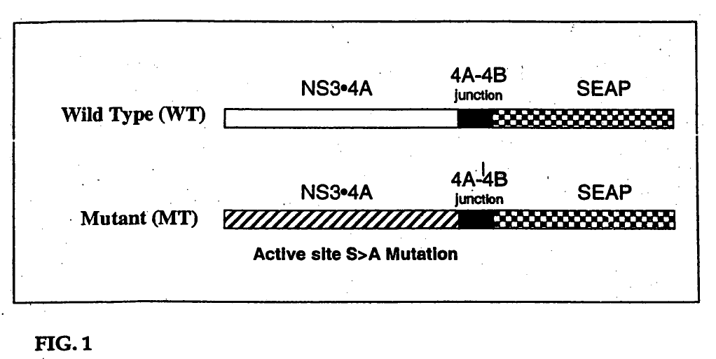

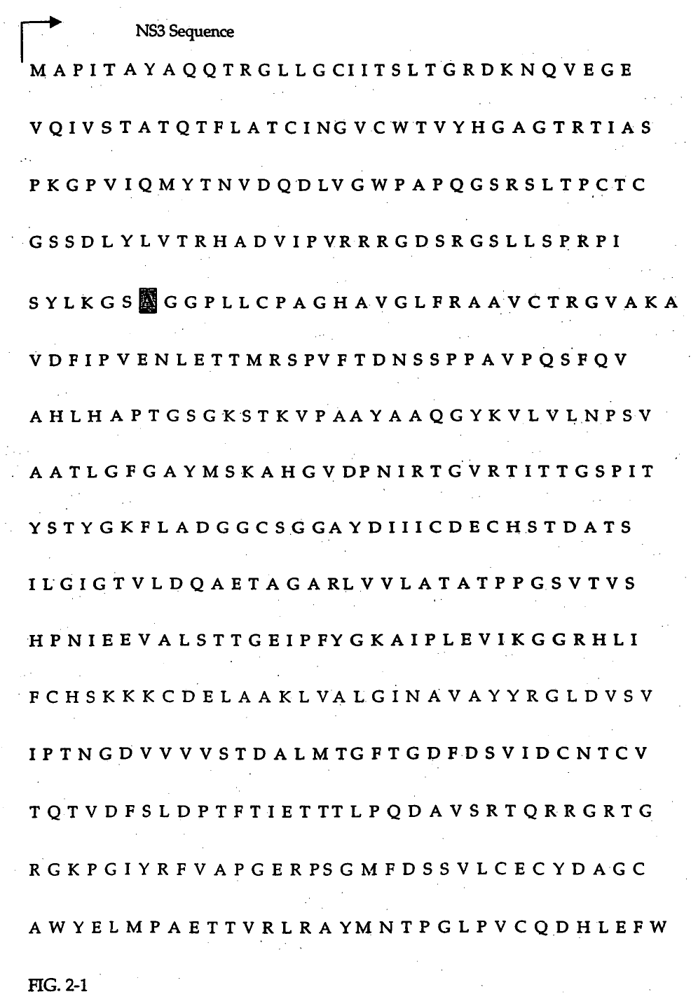

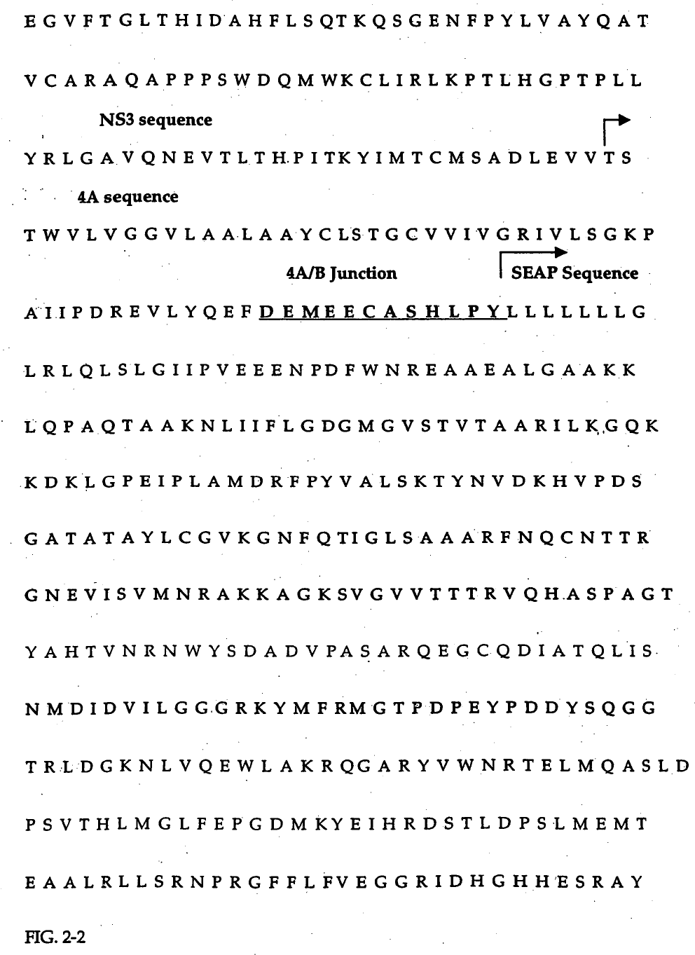

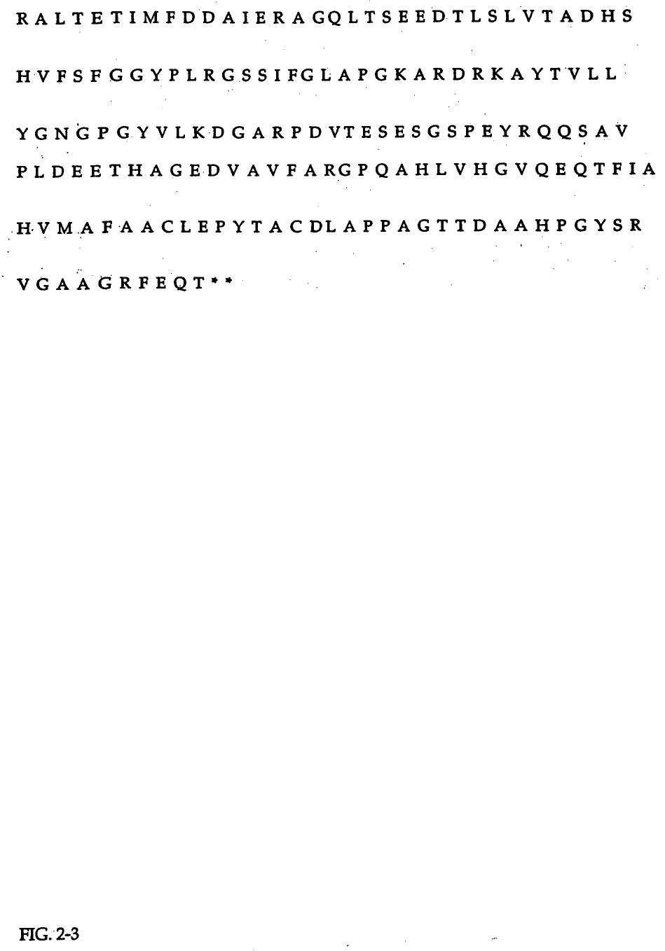

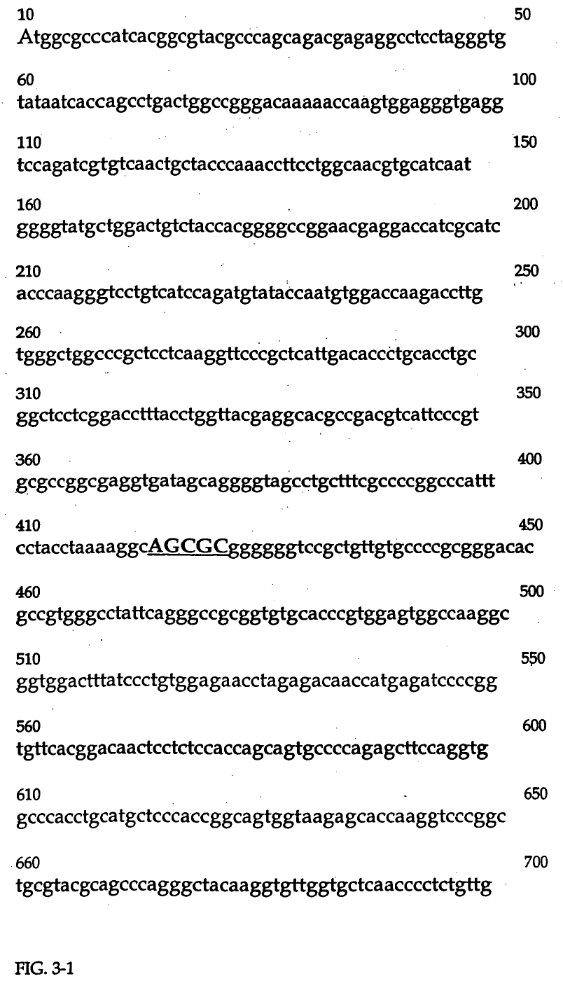

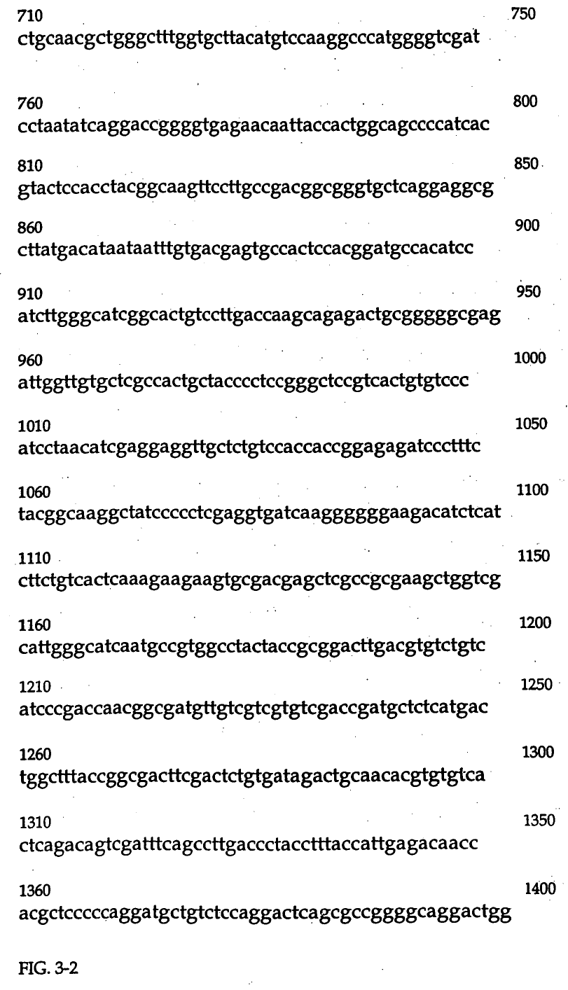

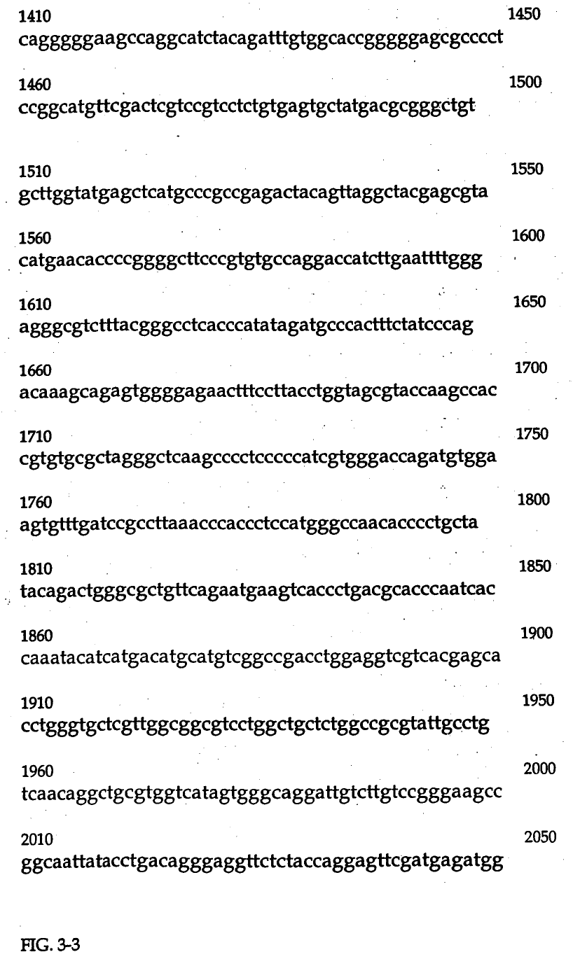

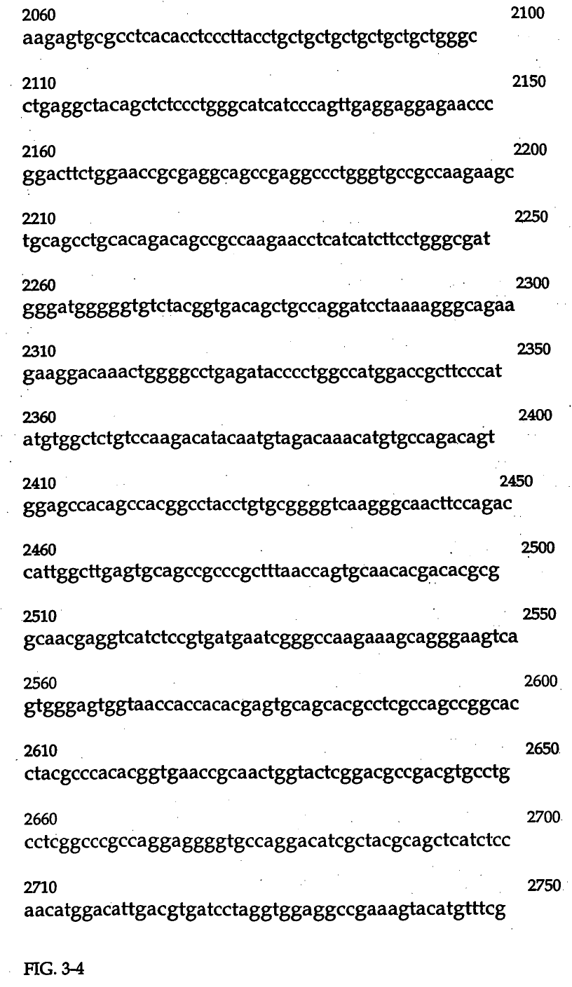

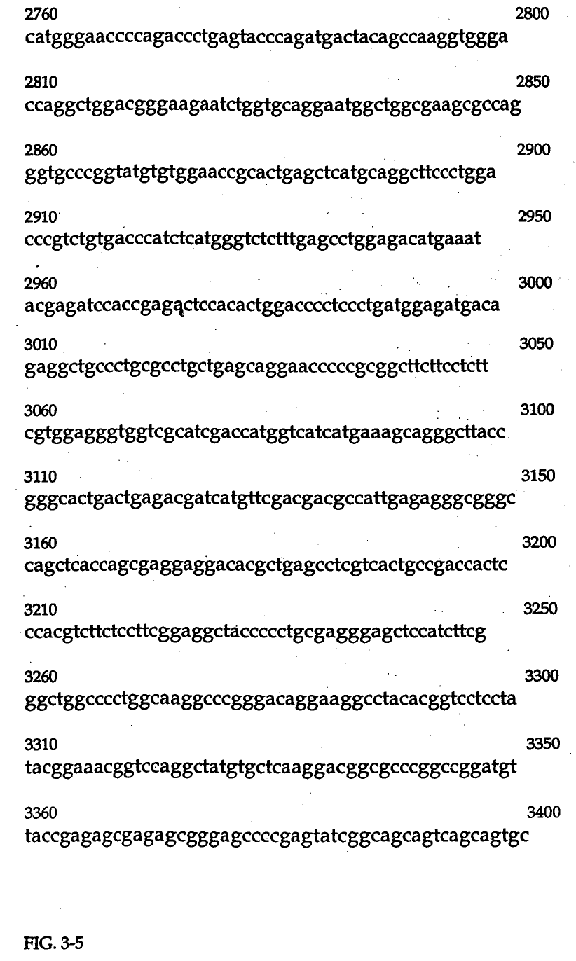

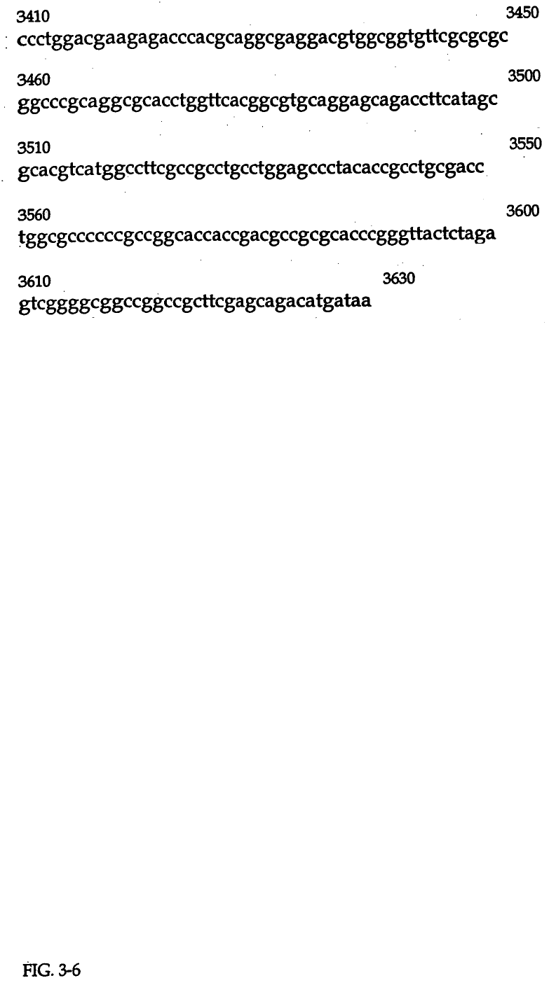

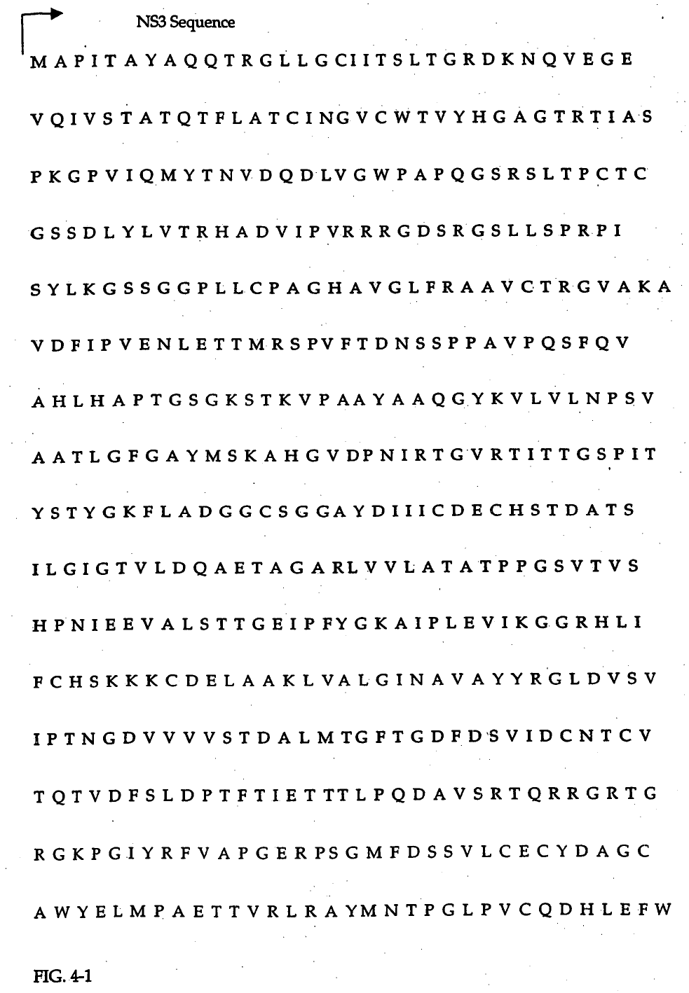

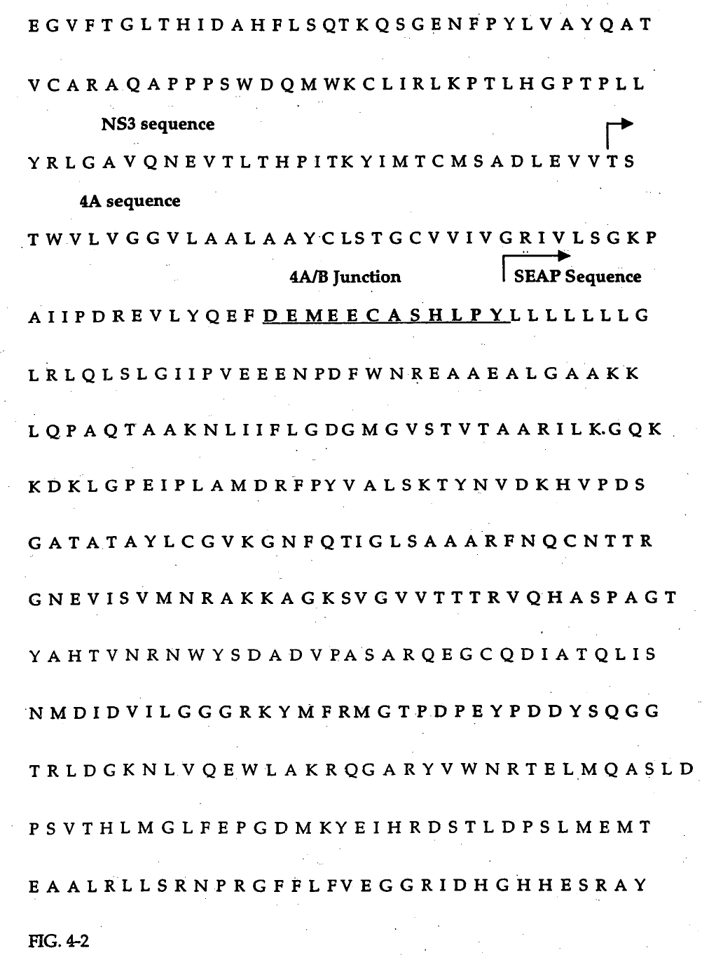

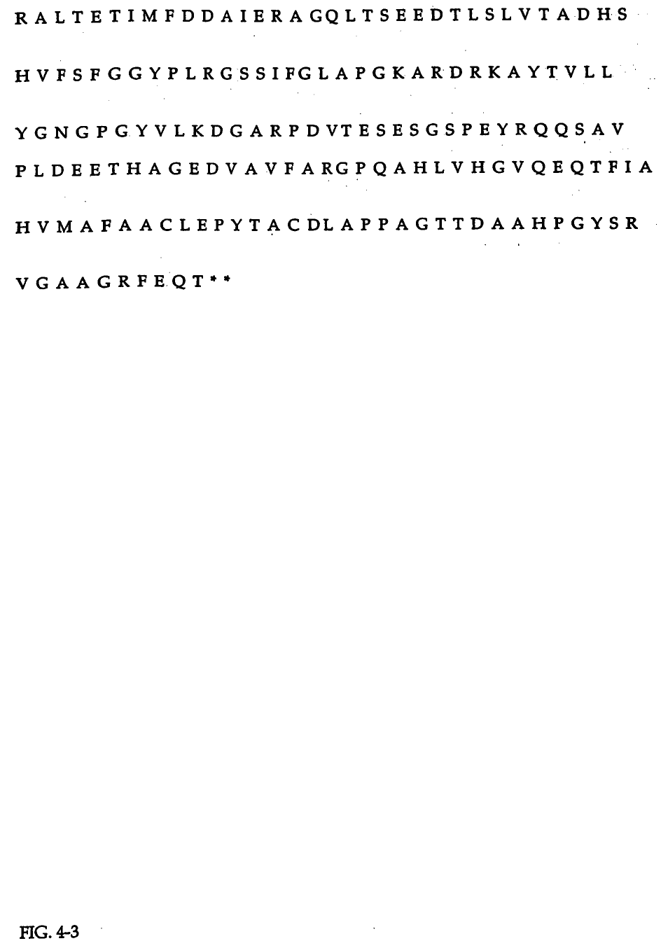

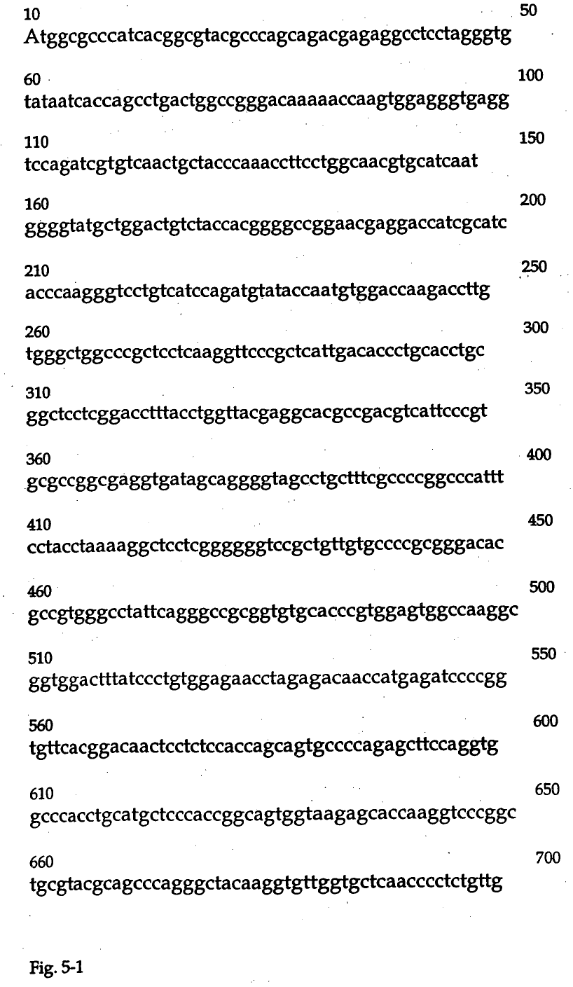

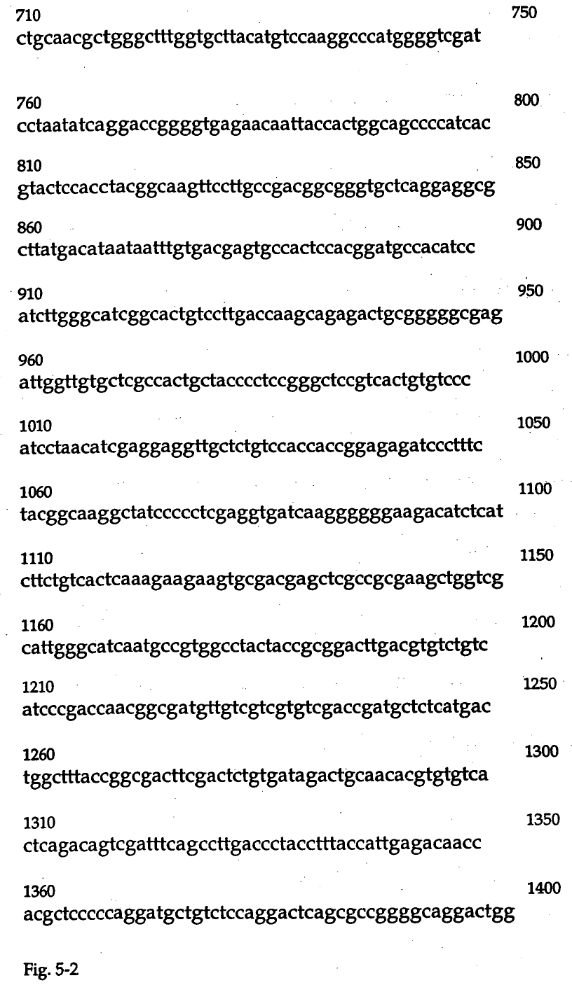

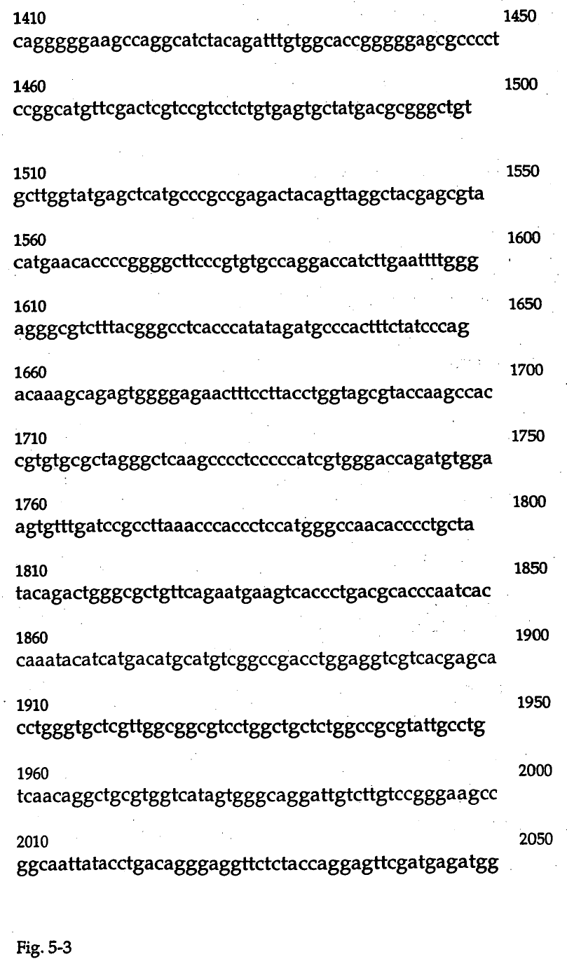

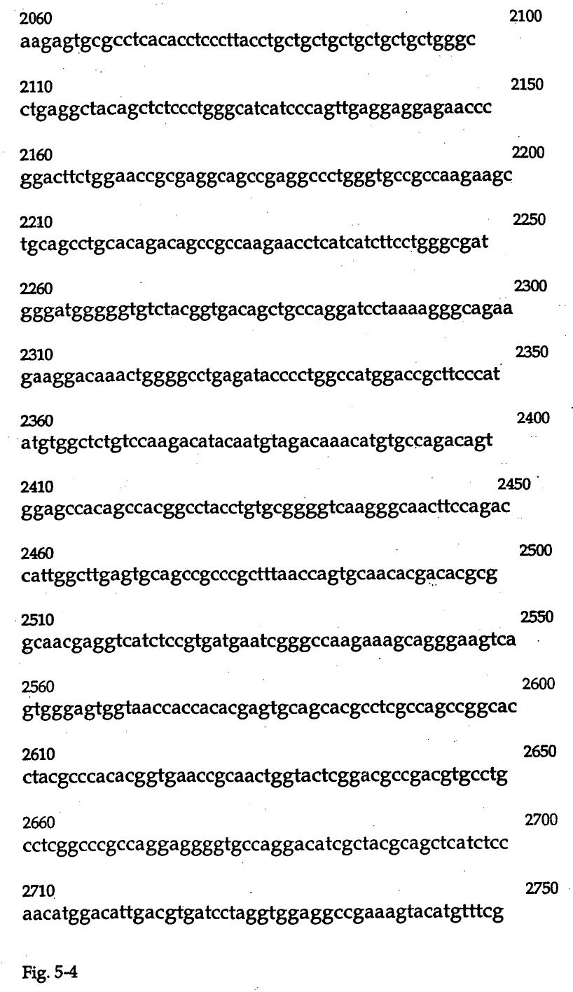

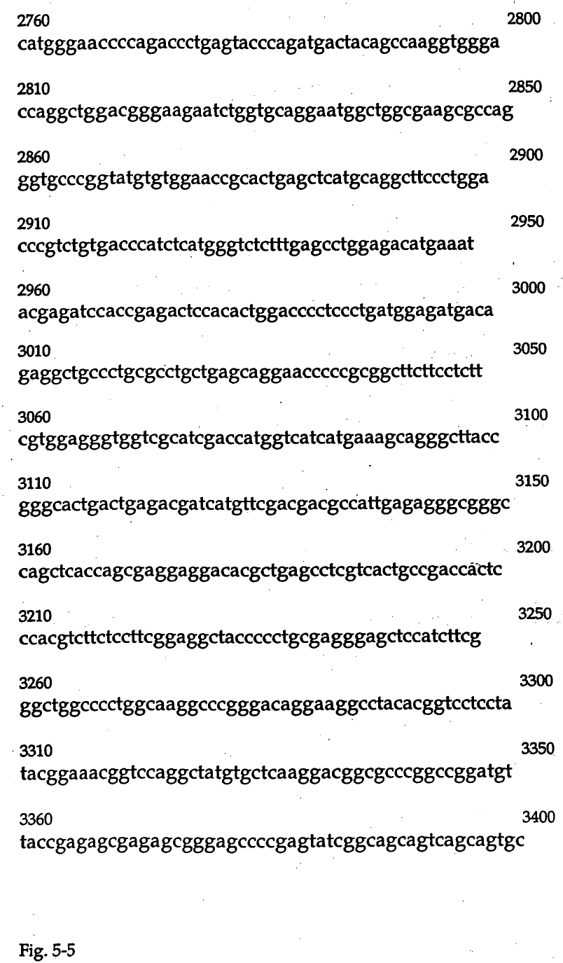

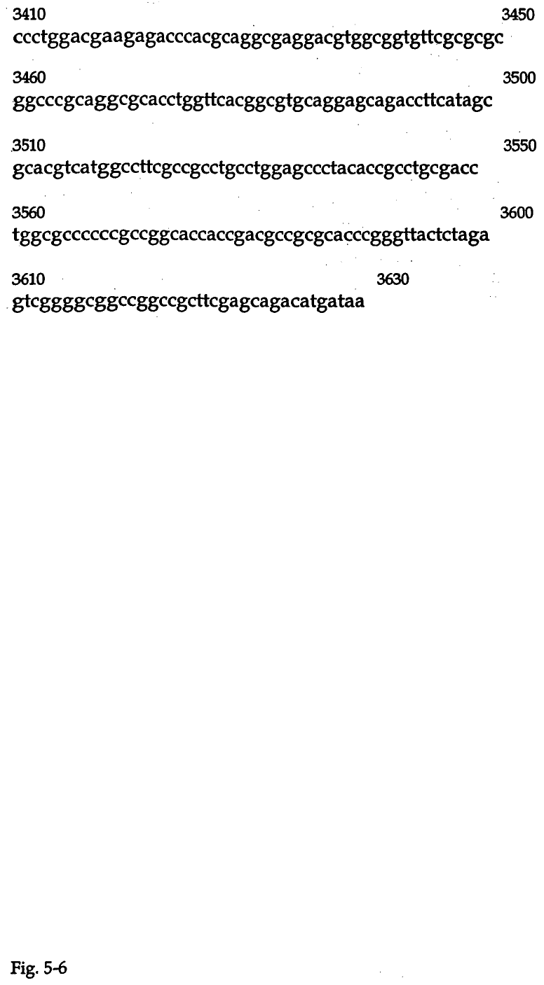

A) Distribution of adenovirus in the infected mice. Detection of HCV NS3 protein in various organs of mice intravenously injected with 10^{9.5} IFU/mice of Ad.HCV.pro.WT.SEAP at 24 hours post infection. H-Heart, Spl-Spleen, Ki-Kidney, Lu-Lungs, Li-Liver. 20 μg of homogenate lysates from the various organs were electrophoresed on 4-12% Bis-Tris protein gels, followed by immuno-detection with an anti-NS3 monoclonal antibody. The 130 kD marker indicates the uncleaved fusion protein, the 70 kD marker indicates the cleaved NS3 protein. The bottom panel indicates the 36 kda Glyceraldehyde 3 phosphate dehydrogenase (GAPDH) protein detected in the same western blots stripped and reprobed with monoclonal antibody directed against mouse GAPDH. GAPDH levels indicate approximately equal amounts of protein loaded in each lane.

B) Demonstration of VX-950 activity in vivo: SCID mice were injected (by tail vein) with 10^{9.5} IFU/mice of either Ad.WT.HCVpro or Ad.MT.HCVpro followed by with or without 300 mg/kg of VX-950 orally twice a day. Serum was collected 24 hours post-infection, followed by chemi-luminescence assay for SEAP. There was a 27 fold reduction in the SEAP secretion in the serum by VX-950, compared to untreated controls. No effect on Mutant HCV protease activity was noticed.

C) Titration of VX-950 in the HCV protease animal model: SCID mice (n=6 per group) were orally dosed with various amounts of VX-950 (as indicated in the figure) and two hours later injected with 10^{9.5} IFU/mice of Ad.WT.HCVpro/mouse by tail vein. Serum was collected from the infected mice 24 hours post-infection and subjected for SEAP chemi-luminescence assay. SEAP levels in the serum of VX-950 treated mice were expressed as % of Vehicle alone treated group. Effective dose 50 (ED50) was calculated as the dose of VX-950 inhibiting the SEAP release in serum by 50% compared to the vehicle alone treated group.

FIGS. 20 and 21: VX-950 protects mice from Liver injury: Six week old SCID mice were injected (by tail vein) with either Ad.WT.HCVpro (10¹¹ IFU/mice A,B and C labeled as WT) or Ad.MT.HCVpro (10¹¹ IFU/mice labeled as MT). Mice (n=8) injected with WT virus were treated either with VX-950 300 mg/kg orally twice a day or with vehicle only for the first three days. Animals were sacrificed after 7 days and the livers were harvested for pathology. The gross appearances of liver from WT protease infected without any drug (No drug) or WT protease infected and treated with VX-950 and MT protease infected are shown in the figure. Hematoxylene, and eosin stained liver sections (400×) from the livers indicating the pathological changes are also shown beneath the respective livers.

FIG. 22 is a schematic representation of the HCV protease animal model. 6 week old SCID mice are injected (by tail vein) with a replication defective adenovirus expressing either wild type HCV protease fused with secreted placental alkaline phosphatase (SEAP) or mutant HCV protease fused with SEAP as control. HCV NS3•4A protease dependent SEAP will be secreted in the blood stream of the infected SCID mice—which can be assayed by chemiluminescence assay.

FIG. 23 depicts a correlation of Steatosis with expression of HCV protease. SCID mice were injected with increasing concentrations of adenovirus expressing wild type HCV protease (ranging from 10⁷ IFU to 10¹¹ IFU/mouse at half log increments). Frozen sections of the livers at 72 hours post infection were stained with Oil Red O. Nuclei were counterstained with hematoxylene. Increasing accumulation of fat in the liver is observed which correlates with increasing expression of SEAP secreted (and thus with HCV protease activity) (20× magnifications are shown in the figure).

FIG. 24 Depicts the correlation of Steatosis with expression of HCV protease. FIG. 24 is the same as FIG. 23 but 40× magnifications are shown. Accumulation of intracellular fat in hepatocytes can be observed.

FIG. 25 depicts that steatosis associated with HCV protease expression can be ameliorated by HCV protease inhibitor VX-950. SCID mice were injected with 10¹¹ IFU/mouse of replication defective adenovirus expressing Wild type HCV protease or mutant HCV protease or SEAP alone (n=6). One group of mice (n=6) injected with adenovirus expressing wild type HCV protease were treated with 300 mg/kg BID via oral route for the initial 3 days. All the mice were sacrificed at 7 days and liver sections were stained with Oil Red O. A significant accumulation of fat in the livers of mice expressing wild type HCV protease compared to either mutant HCV protease or SEAP alone groups (20× magnification).

### DETAILED DESCRIPTION OF THE INVENTION

One embodiment of this invention provides an animal whose liver is targeted for expression of exogenous protein with a construct comprising a promoter operably linked to DNA expressing a protease, wherein the protease is linked to a sequence that is cleavable by the protease, and wherein the sequence is linked to a reporter. The reporter may be present anywhere in the animal, e.g., in the blood, serum, or tissue of the animal, following cleavage by the protease. In one embodiment, the reporter in detectable in the serum.

Any detectable reporter protein may used in this invention. Reporters are typically detected by, for example, chemiluminescence or fluorescence. Typical reporters include secreted alkaline phophatase (SEAP), chloramphenicol acetyltransferase (CAT), luciferase, β-galactosidase, green fluorescent protein (GFP), and horseradish peroxidase. A reporter that may also be used in this invention is a unique protein, i.e., one that does not exist in the animal in its native state, and a suitable antibody or antibody mimic that may be used for detection.

“Animal” as used herein refers to any mammal other than a human. It includes an animal at any age, including embryonic, fetal, newborn, and adult. Animals for use in this invention are available from, e.g., commercial sources. Such animals include but are not limited to lab or other animals, rabbits, rodents (e.g., mice, rats, hamsters, gerbils, and guinea pigs), cows, sheep, pigs, goats, horses, dogs, cats, birds (e.g., chickens, turkeys, ducks, geese), primates (e.g., chimpanzees, monkeys, tamarind, rhesus monkeys). Preferred animals include rats, mice (SCID, etc.), dogs, monkeys. More preferred animals include small animals, such as mice or rats. Most preferably, the mammal is a mouse.

Another embodiment of this invention provides a non-human mammal whose liver is targeted for expression of exogenous protein with a system comprising an operably linked promoter and DNA encoding a protein whose expression causes liver damage. More particularly, the animal is a non-transgenic, non-human animal. Such liver damage may be assessed by, e.g., examining liver morphology, histology, and/or enzyme levels.

As demonstrated herein, the animal models of this invention have livers that are characterized by steatosis. The provided models may also be used to illustrate the fatty liver of HCV infection.

Accordingly, another embodiment of this invention provides a non-human mammal whose liver is targeted for expression of exogenous protein with a system comprising an operably linked promoter and DNA encoding a protein whose expression causes steatosis. Such liver damage may be assessed by, e.g., examining liver morphology, histology, and/or enzyme levels.

The liver damage obtained in an animal model of this invention includes steatosis. Accordingly, this invention also provides a model for diseases and conditions such as NAFLD, nonalcoholic steatohepatitis (NASH), alcoholic steatosis, or Reye's syndrome. These animal models may be used in assays for identifying compounds that modulate steatosis and diseases, disorders, or conditions involving steatosis including, but not limited to, NAFLD, NASH, alcoholic steatosis, and Reye's Syndrome.

Any protein may be employed in this invention, including, but not limited to, enzymes, structural proteins, mammalian proteins, viral proteins, bacterial proteins, and fungal proteins. Preferred proteins include enzymes such as, e.g., proteases, kinases, and esterases. All native, wild-type, and mutant-type DNA and proteins may be utilized in embodiments of this invention.

Embodiments of this invention that involve a protease may employ any protease, including mammalian, viral, fungal, and bacterial proteases.

As recognized by skilled practitioners, proteases may be classified as a serine protease, cysteine protease, aspartic protease, or metalloprotease. All such proteases may be used in an embodiment of this invention. Examples of proteases include, but are not limited to, cathepsins (e.g., cathepsin-B, cathepsin-D, or cathepsin-G), elastase, thrombin, plasmin, C-1 esterase, C-3 convertase, urokinase, plaminogen activator, acrosin, β-lactamase, D-alanine-D-alanine carboxypeptidase, chymotrypsin, trypsin, kallikreins, renin, pepsin, angiotensin converting enzyme, enkephalinase, pseudomonas elastase, leucine aminopeptidase, chymotrypsin, trypsin, elastase, subtilisin, bromelain, papain, thermolysin, caspases (caspase-1, -2, -3, -4, -5, -6, -7, -8, -9, and -10).

Preferred proteases for use in this invention include those from viruses, such as retroviruses (e.g., HIV, HTLV, and Lenti), picoma viruses (e.g., polio), flaviruses (e.g., HCV and other pestiviruses such as BVDV), plant viruses (e.g., capilloviruses), togoviruses (e.g., sindbis), parvo viruses, and adenoviruses. In particular, such viral proteases include, but are not limited to, a protease from a Hepatitis virus (e.g., Hepatitis A, Hepatitis B, Hepatitis C, Hepatitis E, Hepatitis H, or Hepatitis G), a HIV protease, a picomavirus protease, or a Herpes virus (e.g., herpes simplex virus, cytomegalovirus, Epstein-Barr virus, varicella-zoster virus, Kaposi's sarcoma virus) protease.

In one embodiment of this invention, the protease is a Hepatitis C protease. Preferably, the Hepatitis C protease is HCV NS3•4A protease.

The gene systems (i.e., expression constructs that encode e.g., the HCV-SEAP fusion protein) of this invention may be delivered to the liver by using a viral vector, direct injection into the liver, intravenous injection, or non-viral gene transfer. Delivery may also be accomplished using conjugation to non-infectious adenovirus particles, gold particles, lipids, or any non-viral gene transfer.

In one preferred embodiment disclosed herein, adenovirus was used as a vehicle for delivery to the liver. In SCID mice, HCV protease dependent SEAP secretion in the serum of SCID mice infected with Adenovirus expressing HCV protease fused with SEAP was demonstrated. In a preferred embodiment the expression construct is delivered to the liver by using the adenovirus vector described herein.

Practice of this invention involves replication of a gene system (i.e., expression construct), and therefore production of a protein. Any technique leading to the required replication is encompassed by this invention. Typically, such replication is effected by a promoter. Promoters useful for practicing this invention include a CMV, RSV, SV4O, or an albumin promoter. Other techniques are also encompassed by this invention, e.g., IRES elements.

In the present invention, the adenovirus comprises an expression construct that contains the HCV-encoding protease fused to the SEAP reporter, and the expression construct is under to control of a suitable promoter that drives the expression of the HCV protease-SEAP fusion protein. The promoter is typically a heterologous promoter is inserted in such a manner that it is operably linked to allow for the expression of the fusion protein.

The term “expression construct” or “expression vector” is meant to include any type of genetic construct containing a nucleic acid coding for gene products (e.g., fusion proteins of HCV and SEAP) in which part or all of the nucleic acid encoding sequence is capable of being transcribed. Preferably, the transcript is translated into a protein. In certain embodiments, expression includes both transcription of a gene and translation of mRNA into a gene product.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator or repressor proteins.

The particular promoter employed to control the expression of a nucleic acid sequence of interest is not believed to be important, so long as it is capable of directing the expression of the nucleic acid in the targeted cell. Thus, in preferred embodiments, it is preferable to position the nucleic acid coding region adjacent to and under the control of a promoter that is capable of being expressed in a liver cell. Generally speaking, such a promoter might include either a human or viral promoter.

In various embodiments, the human cytomegalovirus (CMV) immediate early-gene promoter, the SV40 early promoter, the Rous sarcoma virus long terminal repeat, β-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well known properties, the level and pattern of expression of the protein of interest following transfection or transformation can be optimized. Inducible promoter systems may be used in the present invention, e.g., inducible ecdysone system (Invitrogen, Carlsbad, Calif.), which is designed to allow regulated expression of a gene of interest in mammalian cells.

In one embodiment of this invention employs a promoter, preferably CMV. In mammalian cells, the CMV immediate early promoter is often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. Liver-specific promoters includes, e.g., IGFBP-1 promoter, the Cx32 gene (Piechocki et al., Carcinogenesis, Vol. 20, No. 3, 401-406, March 1999) is known to have a liver-specific promoter, numerous other liver-specific genes are known to those of skill in the art and promoter elements from such genes may be employed to achieve tissue-specific expression to generate the animal models described herein.

Another regulatory element contemplated for use in the present invention is an enhancer. These are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization. Enhancers useful in the present invention are well known to those of skill in the art and will depend on the particular expression system being employed (Scharf D et al Results Probl Cell Differ 20: 125-62, 1994; Bittner et al Methods in Enzymol 153: 516-544, 1987).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites (Pelletier and Sonenberg, Nature, 334:320-325, 1988). IRES elements from two members of the picornavirus family (poliovirus and encephalomyocarditis) have been described (Pelletier and Sonenberg, 1988 supra), as well an IRES from a mammalian message (Macejak and Sarnow, Nature, 353:90-94, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

There are a number of ways in which expression constructs may introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, In: Rodriguez R L, Denhardt D T, ed. Vectors: A survey of molecular cloning vectors and their uses. Stoneham: Butterworth, 467 492, 1988; Nicolas and Rubenstein, In: Vectors: A survey of molecular cloning vectors and their uses, Rodriguez & Denhardt (eds.), Stoneham: Butterworth, 493 513, 1988; Baichwal and Sugden, In: Gene Transfer, Kucherlapati R, ed., New York, Plenum Press, 117 148, 1986; Temin, In: gene Transfer, Kucherlapati (ed.), New York: Plenum Press, 149 188, 1986). The first viruses used as gene vectors were DNA viruses including the papovaviruses (simian virus 40, bovine papilloma virus, and polyoma) (Ridgeway, 1988 supra; Baichwal and Sugden, 1986 supra) and adenoviruses (Ridgeway, 1988 supra; Baichwal and Sugden, 1986 supra). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals.

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984; Benvenisty and Neshif (Proc. Nat. Acad. Sci. USA, 83:9551-9555, 1986).

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

Also provided by the present invention is a cell or cells obtained from any mammal containing a gene system of this invention. For example, a model animal of the present invention is generated as described herein. Primary liver cells from that animal are subsequently isolated using techniques known to those of skill in the art (see e.g., Culture of Animal Cells, 4^{th} Edn., Ed. Freshney, 2000, Publ. Wiley-Liss, Inc.) The primary cells may then be subcultured and cell lines may thus be generated (see e.g., Chapters 11 and 12 of Culture of Animal Cells, Freshney, 2000).

This invention further provides a hepatocyte cell or hepatocyte cell line comprising a gene system of this invention. More particularly, the invention is directed to a hepatocyte cell or cell line that has been transformed or transfected with an expression construct as described herein. In particularly preferred embodiments, the expression construct encodes an HCV protease fused to a reporter such as a SEAP reporter. Methods and compositions for the transformation of cells are well known to those of skill in the art and may involve any technique routinely employed to modify a cell line with an exogenous nucleic acid sequence.

Also provided by this invention is a viral vector comprising a gene system of this invention for the expression of an expression construct that comprises an HCV protease fused to e.g., SEAP. More particularly, the present invention is directed to an adenoviral vector that comprises an expression construct that encodes such a fusion protein.

Also provided by this invention is a process for producing a mammalian model for protease activity, the method comprising providing a mammal, and delivering to the mammal a gene system (i.e., an expression construct) that comprises A) a promoter, B) DNA encoding a protease, and C) DNA encoding a reporter, wherein A, B, and C are operably linked, and wherein a presence of reporter activity is indicative of protease activity.

Also provided by this invention is a process for producing a mammalian model for liver damage, the method comprising providing a mammal, and delivering to the mammal an expression construct comprising a promoter and DNA encoding a protein whose expression causes hepatocyte damage.

These process embodiments of the invention encompass any of the animals, gene systems, expression constructs and/or methods disclosed herein. The processes may comprise a further step of maintaining the mammal for a sufficient time for, e.g., damage to develop in the liver of the mammal.

In vitro cell-based assay systems have been reported either using HCV protease fused to the secreted placental alkaline phosphatase (SEAP) in COS-7 cells (Y.-G. Cho et al., J. Virol. Methods, 72, pp. 109-115 (1998) or green fluorescent protein (GFP) fused to SEAP protein in COS-7 and in HCV replicon cells (J.-C. Lee et al., Analyt. Biochem., 316, pp. 162-170 (2003)). These methods are not however applicable in vivo.

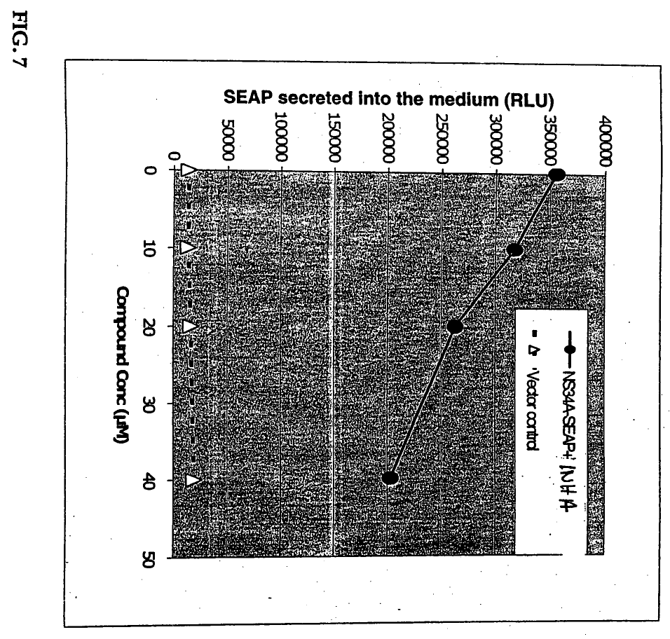

The animals provided herein are useful as in vivo models of protease activity. The utility of the HCV protease animal model was demonstrated by inhibiting the secretion of SEAP into the serum of mice by administration of a HCV protease inhibitor (see, e.g., FIG. 7). Modulation of protease activity may be tested or monitored by measuring or detecting reporter expression.

Accordingly, one embodiment of this invention provides a method for testing an agent which augments or inhibits protease activity, the method comprising:
a) providing a mammal according to any of the embodiments herein;
b) administering the agent to the mammal; and
c) evaluating the effect of the agent on the reporter expression.

Depending on the protease being expressed in this animal model, various agents and compounds may be screened for their effectiveness in enhancing or inhibiting the activity of the protease. This method is particularly useful for testing an agent's effectiveness as an anti-HCV therapy.

HCV protease dependent liver pathology in mice infected with adenovirus expressing HCV protease fused with SEAP was also demonstrated. The utility of the HCV protease liver damage component of this model was demonstrated by showing that a HCV protease inhibitor could protect mice from liver damage associated with the wild type HCV protease expressing SEAP in the adenovirus infection.

Liver injury is commonly seen in HCV patients. To date, it is mostly attributed to the host mediated immune response directed against the virus. However, aggressive course of HCV infections is seen in HIV coinfected patients and immuno compromised patients. The models of this invention may be useful to study the pathogenesis of HCV by itself in the absence of an immune system. Understanding the disease mechanism may result in novel ways to interfere in the disease process and design and develop rational therapies for HCV mediated liver injury. The steatosis observed in the WT HCV protease expressing mice liver suggests a role of HCV protease in pathogenesis of hepatitis C.

Accordingly, one embodiment of this invention also provides a method for assessing an agent which augments or inhibits liver damage, comprising:
a) providing a mammal according to any of the embodiments herein;
b) administering the agent to the mammal; and
c) evaluating the effect of the agent on the damage.

Any class of agent or compound may be tested and/or screened in this liver damage assay. For example, any agent that has been implicated in causing liver damage or in treating and/or preventing liver damage may be tested. These agents include, but are not limited to, protease inhibitors, caspase inhibitors (e.g., ICE inhibitors, caspase-3 inhibitors, caspase-7 inhibitors, etc.), kinase inhibitors (e.g., serine and threonine protein kinase inhibitors), IMPDH inhibitors, phosphatase inhibitors, protease inhibitors, esterase inhibitors, lipase inhibitors, cytokine inhibitors (e.g., inhibitors of TNF-alpha, TGF-beta), apoptosis mediators and/or inhibitors (e.g., PARP), antibodies (or fragments thereof), Fab fragments, and antibody-like peptides or proteins (or fragments thereof). This method is particularly useful for testing an agent's effectiveness to treat or prevent liver damage, including steatosis.

Accordingly, this invention provides methods for inhibiting liver damage, steatosis, NAFLD, NASH, alcoholic steatosis, or Reye's syndrome by administering a compound identified according to a method of this invention.

The agents or candidate substance being tested for therapeutic efficacy against liver damage may be a protein or fragment thereof, a small molecule inhibitor, or even a nucleic acid molecule. It may prove to be the case that the most useful pharmacological compounds for identification through application of the screening assay will be compounds that are structurally related to other known modulators of obesity. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential.

Accordingly, the active compounds may include fragments or parts of naturally-occurring compounds or may be found as active combinations of known compounds which are otherwise inactive. Accordingly, the present invention provides screening assays to identify agents which inhibit or otherwise treat the indicia of obesity. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known agents that are used in the treatment of liver disease.

“Effective amounts” in certain circumstances are those amounts effective to reproducibly alter a given indicator of liver disease.

Treatment of animals with test compounds will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood, cerebrospinal fluid (CSF) or lymph supply and intratumoral injection.

Determining the effectiveness of a compound in vivo may involve a variety of different criteria. Such criteria include, but are not limited to, survival, inhibition or prevention of inflammatory response, increased activity level, improvement in immune effector function and improved food intake.

As demonstrated herein, the HCV protease inhibitor VX-950 is effective at ameliorating steatosis. Accordingly, another embodiment of this invention provides a method for inhibiting steatosis comprising administering to a patient in need thereof an effective amount of a protease inhibitor. This invention also provides a method for treating or preventing a disease, condition, or disorder that has steatosis as a symptom. Such diseases include, but are not limited to NAFLD, NASH, alcoholic steatosis, and Reye's syndrome.

VX-950 is a competitive, reversible peptidomimetic HCV NS3/4A protease inhibitor with a steady state binding constant (ki*) of 3 nM (and with a Ki of 8 nM) (see poster presented by Perni, et al. at AASLD meeting, Boston, October and 2003; WO 02/18369).

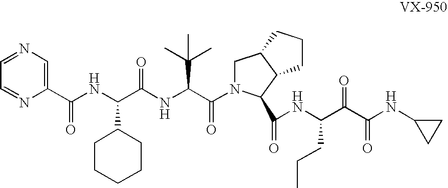

Other protease inhibitors may also be used in the present invention. For example, chiral mixtures of VX-950 may be used. In certain embodiments, compounds used may be mixtures of the D- and L-isomers at the N-propyl-side chain of the following structure:

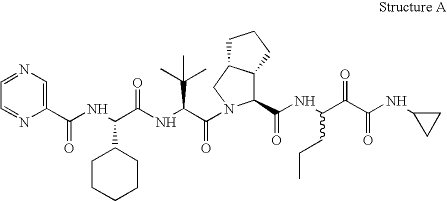

The compound used in the experiments described in the examples herein, designated as “VX-950”, exists as a mixture of epimers at the n-propyl side chain. This compound is represented below as structure A. It would be recognized that structure A depicts a diastereomeric mixture of D- and L-isomers at the n-propyl side chain. Other agents generated through rational drug design using e.g., VX-950 or the compound of Structure A as a starting compound may be tested for their activity as protease inhibitors. In addition, those of skill in the art are aware of numerous other protease inhibitors that could be tested in the methods of the present invention. Exemplary such inhibitors include HCV protease inhibitors that have been described in PCT publication Nos. WO 02/18369, WO 02/08244, WO 00/09558, WO 00/09543, WO 99/64442, WO 99/07733, WO 99/07734, WO 99/50230, WO 98/46630, WO 98/17679 and WO 97/43310, U.S. Pat. No. 5,990,276, M. Llinas-Brunet et al., Bioorg. Med. Chem. Lett., 8, pp. 1713-18 (1998); W. Han et al., Bioorg. Med. Chem. Lett., 10, 711-13 (2000); R. Dunsdon et al., Bioorg. Med. Chem. Lett., 10, pp. 1571-79 (2000); M. Llinas-Brunet et al., Bioorg. Med. Chem. Lett., 10, pp. 2267-70 (2000); and S. LaPlante et al., Bioorg. Med. Chem. Lett., 10, pp. 2271-74 (2000)). These and other compositions comprising protease inhibitors may be used in methods of treating steatosis.

Combination therapy of steatosis and or other liver disease is alos contemplated. Such combination therapy methods of this invention may also involve administration of another component comprising an additional agent selected from an immunomodulatory agent; an antiviral agent; an inhibitor of HCV protease; an inhibitor of another target in the HCV life cycle; an inhibitor of internal ribosome entry, a broad-spectrum viral inhibitor; another cytochrome P-450 inhibitor; hepatoprotective agents; steatosis inhibitors; or combinations thereof. See, WO 02/18369.

Accordingly, in another embodiment, this invention provides a method comprising administering a protease inhibitor, and another anti-viral agent, preferably an anti-HCV agent. Such anti-viral agents include, but are not limited to, immunomodulatory agents, such as α-, β-, and γ-interferons, pegylated derivatized interferon-α compounds, and thymosin; other anti-viral agents, such as ribavirin, amantadine, and telbivudine; other inhibitors of hepatitis C proteases (NS2—NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenylic acid and derivatives thereof, and including, but not limited to VX-497, VX-148, and/or VX-944); or combinations of any of the above.

The following definitions are used herein (with trademarks referring to products available as of this application's filing date).
“Peg-Intron” means PEG-Intron®, peginteferon alfa-2b, available from Schering Corporation, Kenilworth, N.J.;
“Intron” means Intron-A®, interferon alfa-2b available from Schering Corporation, Kenilworth, N.J.;
“ribavirin” means ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition; also available as Rebetol® from Schering Corporation, Kenilworth, N.J., or as Copegus® from Hoffmann-La Roche, Nutley, N.J.;
“Pagasys” means Pegasys®, peginterferon alfa-2a available Hoffmann-La Roche, Nutley, N.J.;
“Roferon” mean Roferon®, recombinant interferon alfa-2a available from Hoffmann-La Roche, Nutley, N.J.;
“Berefor” means Berefor®, interferon alfa 2 available from Boehringer Ingelheim Pharmaceutical, Inc., Ridgefield, Conn.;
Sumiferon®, a purified blend of natural alpha interferons such as Sumiferon available from Sumitomo, Japan;
Wellferon®, interferon alpha n1 available from Glaxo_Wellcome Ltd., Great Britain;
Alferon® (, a mixture of natural alpha interferons made by Interferon Sciences, and available from Purdue Frederick Co., CT;
The term “interferon” as used herein means a member of a family of highly homologous species-specific proteins that inhibit viral replication and cellular proliferation, and modulate immune response, such as interferon alpha, interferon beta, or interferon gamma. The Merck Index, entry 5015, Twelfth Edition. Interferons are also described in WO 02/018369. Any of these interferons may be used in the embodiments of this invention. HCV NS3/4A serine protease blocks phosphorylation and effector action of interferon regulatory factor-3 (IRF-3; see Foy et al., Science 300, 1145-1148). IRF-3 is a key signaling molecule and therefore the action of HCV protease on this molecule may lead to the deleterious effects of HCV infection Inhibition of HCV protease may restore the function of IRF-3 and be of therapeutic value in the treatment of HCV infection. The animal models of the present invention may therefore be employed to test for the efficacy of agents that will ameliorate the action of HCV protease on IRF-3 signaling.

According to a preferred embodiment of the present invention, the interferon is α-interferon. According to another embodiment, a the present invention utilizes natural alpha interferon 2a. Or, the present invention utilizes natural alpha interferon 2b. In another embodiment, the present invention utilizes recombinant alpha interferon 2a or 2b. In yet another embodiment, the interferon is pegylated alpha interferon 2a or 2b. Interferons suitable for the present invention include:
(a) Intron,
(b) Peg-Intron,
(c) Pegasys,
(d) Roferon,
(e) Berofor,
(f) Sumiferon,
(g) Wellferon,
(h) consensus alpha interferon available from Amgen, Inc., Newbury Park, Calif.,
(i) Alferon;
(j) Viraferon®;
(k) Infergen®.

As is recognized by skilled practitioners, oral administration is preferred in therapeutic regmimens. Interferon is not typically administered orally. Nevertheless, nothing herein limits the methods or compositions of this invention to any specific dosage forms or regimen. Thus, each component of the methods and compositions of this invention may be administered separately, together, or in any combination thereof.

A method according to this invention may also comprise administering a cytochrome P450 monooxygenase inhibitor. CYP inhibitors may be useful in increasing liver concentrations and/or increasing blood levels of compounds that are inhibited by CYP.

The advantages of improving the pharmacokinetics of a drug (e.g., by administering a CYP inhibitor) are well accepted in the art. By administering a CYP inhibitor, this invention provides for decreased metabolism of the protease inhibitor, VX-950. The pharmacokinetics of the VX-950 are thereby improved. The advantages of improving the pharmacokinetics of a drug are well accepted in the art. Such improvement may lead to increased blood levels of the protease inhibitor. More importantly for HCV therapies, the improvement may lead to increased concentrations of the protease inhibitor in the liver.

In a method of this invention, the amount of CYP inhibitor administered is sufficient to increase the blood levels of VX-950 as compared to the blood levels of this protease inhibitor in the absence of a CYP inhibitor. Advantageously, in a method of this invention, an even further lower dose of protease inhibitor may be therefore used (relative to administration of a protease inhibitor alone).

CYP inhibitors include, but are not limited to, ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole.

Methods for measuring the ability of a compound to inhibit cytochrome P450 monooxygenase activity are known (see U.S. Pat. No. 6,037,157 and Yun, et al. Drug Metabolism & Disposition, vol. 21, pp. 403-407 (1993).

A CYP inhibitor employed in this invention may be an inhibitor of only one isozyme or more than one isozyme. If the CYP inhibitor inhibits more isozyme, the inhibitor may nevertheless inhibit one isozyme more selectively than another isozyme. Any such CYP inhibitors may be used in a method of this invention.

Embodiments of this invention may employ compositions comprising a compound, e.g., a protease inhibitor such as VX-950, or a pharmaceutically acceptable salt thereof. As would be recognized, such compositions typically comprise a pharmaceutically acceptable carrier and may comprise additional agents as described herein (e.g., a CYP inhibitor). Each component may be present in individual compositions, combination compositions, or in a single composition.

If pharmaceutically acceptable salts of the compounds are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions are formulated for pharmaceutical administration to a mammal, preferably a human being.

Such pharmaceutical compositions of the present invention (as well as compositions for use in methods, compositions, kits, and packs of this inventions) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously.

Sterile injectable forms of the compositions of and according to this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In compositions used in accordance with this invention (i.e., compositions used in methods, kits, compositions, or packs of this invention), the compound and any optional additional agent should be present at dosage levels of between about 10 to 100%, and more preferably between about 10 to 80% of the dosage normally administered in a monotherapy regimen.

The pharmaceutical compositions may be orally administered in any orally acceptable dosage form including, but not limited to, capsules, tablets, pills, powders, granules, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Acceptable liquid dosage forms include emulsions, solutions, suspensions, syrups, and elixirs.

Alternatively, the pharmaceutical compositions may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of, and according to, this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

As is recognized in the art, pharmaceutical compositions may also be administered in the form of liposomes.

Preferred are pharmaceutical compositions of, and according to, this invention formulated for oral administration.

According to a preferred embodiment, a protease inhibitor is (preferably, VX-950) is present in an amount effective to decrease the steatosis in a sample or in a patient.

Dosage levels of between about 0.01 and about 100 mg/kg body weight per day, preferably between about 0.5 and about 75 mg/kg body weight per day of a compound (e.g., VX-950) are useful in the methods of this invention. For a CYP inhibitor, the dosage levels of between about 0.001 to about 200 mg/kg body weight per day, would be typical. More typical would be dosage levels of between about 0.1 to about 50 mg/kg or about 1.1 to about 25 mg/kg per day. Typically, the pharmaceutical compositions of, and according to, this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

As recognized by skilled practitioners, dosages of interferon are typically measured in IU (e.g., about 4 million IU to about 12 million IU).

Upon improvement of a patient's condition, a maintenance dose of a compound or composition may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound and the presence or absence and the nature of the additional anti-viral agent in the composition.

For preferred dosage forms of ritonavir, see U.S. Pat. No. 6,037,157, and the documents cited therein: U.S. Pat. No. 5,484,801, U.S. application Ser. No. 08/402,690, and International Applications WO 95/07696 and WO 95/09614).

According to another embodiment, the invention provides a method for treating a patient 1) infected with a virus characterized by a virally encoded NS3/4A serine protease that is necessary for the life cycle of the virus; or 2) suffering from liver damage by administering to said patient a pharmaceutically acceptable composition comprising a compound identified by a method of this invention. A preferred patient is a human being.

In yet another embodiment the present invention provides a method of pre-treating a biological substance intended for administration to a patient comprising the step of contacting said biological substance with a pharmaceutically acceptable composition comprising a compound of this invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

This invention also provides a process for preparing a composition comprising a compound identified by a method of this invention. Another embodiment of this invention provides a process comprises combining a compound identified by a method of this invention and one or more additional agent as described herein.

Pharmaceutical compositions may also be prescribed to the patient in “patient packs” containing the whole course of treatment in a single package, (e.g., a blister pack). Patient packs have an advantage over traditional prescriptions, where a pharmacist divides a patients supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of a composition means of a single patient pack, or patient packs of each formulation, containing within a package insert instructing the patient to the correct use of the invention is a desirable additional feature of this invention.

According to a further aspect of the invention is a pack comprising at least a compound identified by a method according to this invention and an information insert containing directions on the use of the compound. In an alternative embodiment of this invention, the pharmaceutical pack further comprises one or more of additional agents as described herein. The additional agent or agents may be provided in the same pack or in separate packs.

Another aspect of this involves a packaged kit for conducting a method according to this invention, comprising: the material for conducting the method and; and instructions for carrying out drug administration in a manner effective to treat or prevent HCV infection.

Accordingly, this invention provides kits for the simultaneous or sequential administration of a compound identified according to this method (and optionally an additional agent) or derivatives thereof are prepared in a conventional manner. Typically, such a kit will comprise, e.g. a composition of each inhibitor and optionally the additional agent(s) in a pharmaceutically acceptable carrier (and in one or in a plurality of pharmaceutical formulations) and written instructions for the simultaneous or sequential administration.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle or a vial. The present kits will also typically include means for packaging the individual kit components, i.e., the dosage forms, the container means, and the written instructions for use. Such packaging means may take the form of a cardboard or paper box, a plastic or foil pouch, etc.

### General Methodology

VX-950 and may be prepared in general by methods known to those skilled in the art (see, e.g., documents cited herein).

Routine techniques that are known to skilled practitioners may be used to practice this invention. Such techniques may be found in published documents. For example, standard recombinant DNA and molecular cloning techniques are well known in the art. See, e.g., F. M. Ausubel, Current Protocols in Molecular Biology, John Wiley & Sons, Inc., Media, Pa.; Sambrook, J., Fritsch, E. F. and Maniatis, T., Molecular Cloning: A Laboratory Manual; Cold Spring Harbor Laboratory Press: Cold Spring Harbor, 1989, and the literature documents cited in U.S. Pat. Nos. 6,617,156, and 6,617,130, all of which are hereby incorporated by reference.

In order that this invention be more fully understood, the following examples are set forth. These examples are for the purpose of illustration only and are not to be construed as limiting the scope of the invention in any way.

### EXAMPLE 1

### Design and Construction of Reporter Genes

Over lapping PCR was used to fuse cDNA encoding HCV NS3•4A and secreted placental alkaline phosphatase. HCV NS3•4A DNA was PCR amplified using:
a) NS4A U2 5′CAG CAG CAG GTA AGG GAG GTG TGA GGC GCA CTC TTC CAT CTC ATC GAA CTC 3′ as the upper primer with:
b) NS4A L4 5′ TGT CTG TCA TCC CGA CCA ACG3′ as the lower primer. This resulted in a PCR product of 893 bp using pYes2/NS3•4A plasmid as a template. PCR conditions were 94° C. for 45 secs., 50° C. for 45 secs., 72° C. for 1 min.

Similarly, the secreted placental alkaline phosphatase (SEAP) was PCR amplified:
a) SEAP L3 5′AGTG AGA TCT GCGGCCGC TTA TCA TGT CTG CTC GAA GC GG3′ as the lower primer with
b) SEAP U 5′ TCA CAC CTC CCT TAC CTG CTG CTG CTG CTG CTG C3′ as the upper primer with PCR conditions 94° C. 45 secs., 55° C. 45 sec and 72° C. for 1.3 minutes.

The resultant PCR products were gel purified using a Qiaex II gel extraction kit (Cat#20021 Qiagen). The over lap PCR to fuse the PCR products NS3•4A and SEAP was performed using these gel purified products as templates and NS4A L4 & SEAP L3 as primers and PfU polymerase (Stratagene) for amplification.

In an additional embodiment, a further construct was created. In this construct, the NS4A•4B junction of HCV genotype 1b-(DEMEEC-ASHL) was fused in-frame between the gene for HCV NS3•4A and the reporter gene encoding secreted placental alkaline phosphatase (SEAP) using overlapping PCR. HCV NS3•4A was amplified from pYes2-NS3•4A plasmid (Markland et al 1997) at 94° C. for 30 s, 50° C. for 30 s and 72° C. for 60 s with the following PCR primers:
Upper primer: TGTCTGTCATCCCGACCAACG (nt 1193-1213 of NS3 or nt 2211-2231 of pShuttle/NS34A-SEAP (DEMEEC.ASHLPY junction) construct
Lower Primer: CAGCAGCAGCAGCAGGAGGTGTGAGGCGCACTCTTCCATCTCA TCGAACTC (Italics represent SEAP ORF sequence nt 4-19 and the underlined sequence is the NS4A sequence nt 165-142).

Secreted placental alkaline phosphatase (SEAP) was amplified using pSEAP2 (Clontech, Palo Alto, Calif.) at 94° C. for 45 s, 55° C. for 45 s and 72° C. for 90 s with the following PCR primers:

Upperprimer: AGTGAGATCTGCGGCCGCTTATCATGTCTGCTCGAAGCGG (Italics represent the Not I restriction site and the underlined sequence represent SEAP ORF sequence nt 1560-1542).

Lower primer: TGCGCCTCACACCTGCTGCTGCTGCTGCTGCTGGGC (Underlined sequence represent the coding sequence for CASHL which is part of the 4A•4B junction. Italics represent the SEAP ORF sequence nt 4-28).

The 897 bp HCV NS3•4A and the 1591 bp SEAP PCR products were used as templates for overlapping PCR, using TGTCTGTCATCCCGACCAACG as upper primer (nt 1193-1213 of NS3) and CCCACCTTGGCTGTAGTC (spanning nt 709-726 of SEAP ORF or nt 3799-3817 of pShuttle HCV WT/MT NS3•4A-SEAP [DEMEEC.ASHLPY junction]) as lower primer. The PCR product of 1.6 kb size was restriction digested with Sal I and Pvu II (yielding a restriction fragment of 1039 bp size) and cloned into pShuttle HCV WT NS3•4A-SEAP (DEMEEC.ASHLPY junction) and pShuttle HCV MT NS3•4A-SEAP (DEMEEC.ASHLPY junction) clones.

The recombinant clones were confirmed by diagnostic restriction digestion with Hind III (CRL 1830 mouse hepatocytes were transfected with pShuttle HCV WT-NS3•4A-SEAP and pShuttle HCV MT-NS3•4A-SEAP (DEMEEC-ASHL junction) and the expression of HCV protease was tested by western blot after 48 hours post transfection.

pShuttle HCV WT-NS3•4A-SEAP and pShuttle HCV MT-NS3•4A-SEAP (DEMEEC-ASHL Junction) were digested with homing endonucleases (Iceu I and PIsce I) and cloned in pAdenoX (Clontech, Palo Alto, Calif.) as described earlier.

### EXAMPLE 2

### Cloning of PCR Product in Plasmid Vectors

The 2.5 kb over lap PCR product was restriction digested with SalI and BglII and cloned into pYes NS3•4A encoding either the wild type HCV protease (WT) or the mutant (MT) protease containing a serine to alanine mutation in the active site of HCV protease using T4 DNA ligase (New England Biolabs). The 3.7 kb HindIII-NotI cDNA fragment encoding HCV NS3•4A SEAP was cloned into the pCEP4 mammalian expression vector. The resultant clones were subjected to DNA sequencing at the DNA core facility of Vertex Pharmaceuticals Incorporated.

### EXAMPLE 3

### Expression of the HCV-SEAP Reporter Plasmids in Cell Culture

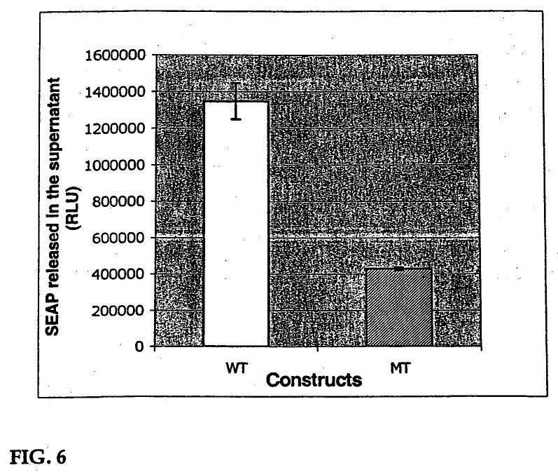

CRL 1830 mouse hepatocytes in 12 well plates were transfected with 2.4 μg of pCEP4 encoding either HCV WT NS3•4A SEAP or SEAP cDNA with lipofectamine2000 (Invitrogen). CRL 1830 mouse hepatocytes in 12 well plates were transfected in duplicates with 2.4 ugm of HCV WT NS3•4A SEAP and HCV MT NS3•4A SEAP. 72 hours post transfection the medium was assayed for SEAP activity. As shown in FIG. 6, cells transfected with the WT HCV Protease construct secreted more SEAP into the media than cells which received the mutant (MT) protease.

In the experiment shown in FIG. 7, CRL 1830 mouse hepatocytes in 12 well plates were transfected with pCEP4 encoding either HCV WT NS3•4A SEAP or SEAP cDNA with lipofectamine2000 (Invitrogen) and treated with various concentrations of HCV protease inhibitor ranging from 0 to 40 μM. 48 hours later the medium was assayed for SEAP activity. FIG. 7 shows the dose dependent inhibition of SEAP secretion an HCV protease inhibitor A.

### EXAMPLE 4

### Cloning of HCV WT and MT NS3•4A SEAP into Adenovirus

As shown in FIG. 8, the NheI-NotI fragment from pCEP4-HCV 34A-SEAP was cloned into pShuttle vector which is a transfer vector for adenovirus. Pshuttle HCV WT and MT NS3•4A SEAP was double digested with PI-SceI and IceuI and cloned into similarly digested pAdeno-X DNA (BDBioscienes). PacI restriction digested pAdeno-HCV WT/MT NS3•4A SEAP DNA was transfected into HEK293 cells (ATCC) using the calcium phosphate method of transfection (Calcium Phosphate transfection reagent, Gibco BRL). Cells were allowed to grow for two weeks until the cytopathic effect of the Adenovirus infection appeared, after which the cells were harvested for recombinant adenovirus. The virus was amplified by subsequently infecting larger numbers of HEK 293 cells. Adenovirus encoding HCV WT/MT NS3•4A SEAP were purified using cesium chloride banding.

### EXAMPLE 5

### Expression of HCV-SEAP in Cells Transduced with Adenovirus Vectors Carrying the HCV WT and MT NS3•4A SEAP Reporter Constructs

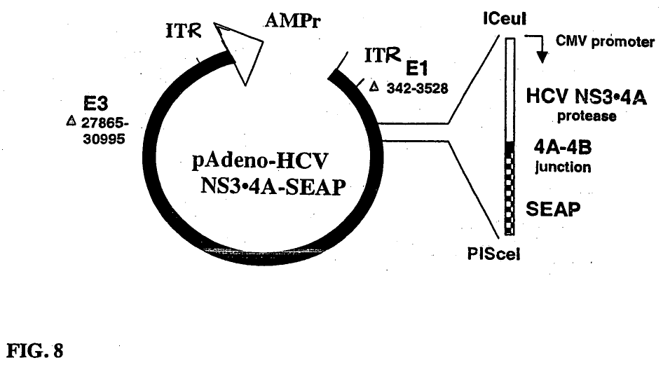

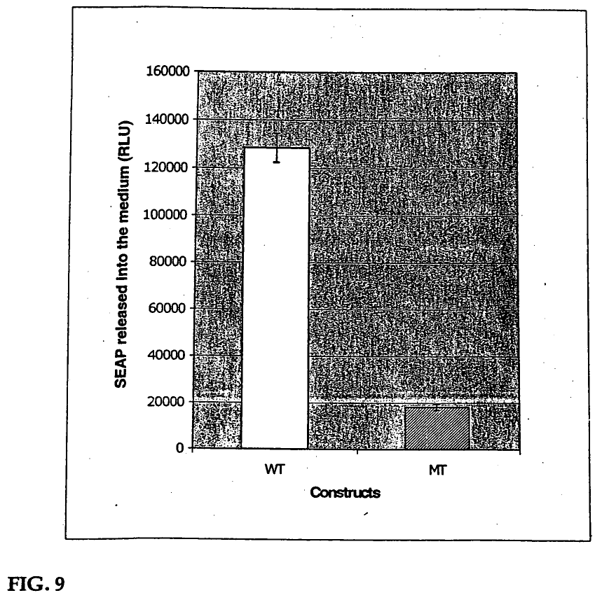

Adenovirus expressing HCV NS3•4A SEAP WT and Mutant were prepared and diluted to an MOI of 20. CRL 1830 mouse hepatocytes 5×10⁵ cells seeded in 12 well plates were used for infection after 24 hours. As shown in FIG. 9, mouse hepatocytes infected with adenovirus carrying WT HCV NS3•4A fused to the SEAP reporter through the NS4A-NS4 junction secreted significantly more SEAP into the media than cells that received the mutant construct.

To test the ability of a HCV protease inhibitor to inhibit HCV protease-dependent secretion of SEAP, inhibitor B was diluted to 80, 40, 20, 10, 5 times its IC50 and 1 ml was added to each well of cells 30 minutes before infection (final concentration was 40,20,10,5,2.5×IC50).

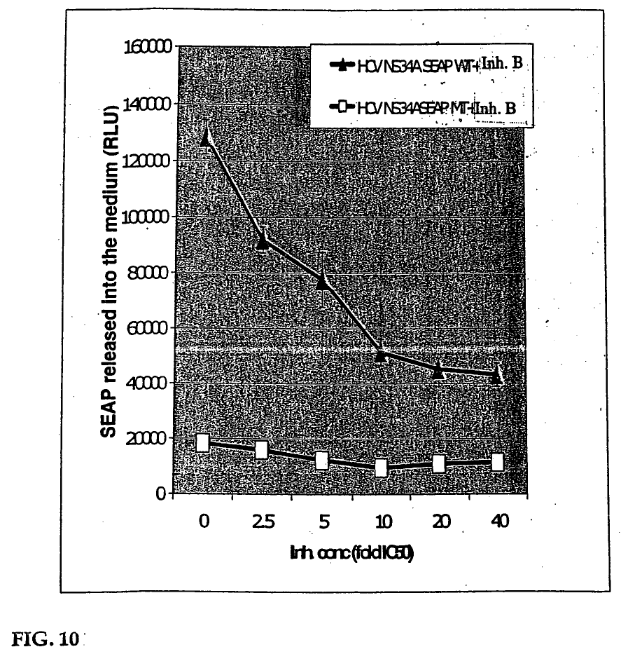

Adenoviruses were diluted (original stock 1.8×10¹²) 1:100 by adding 2 uL in 200 μL and then adding 14 μL to 12.5 ml of medium (this should give an MOI of 20) and 1 ml of medium was added to each well. Cells were incubated for 72 hours and 20 μL of medium was used for SEAP chemiluminescence assay (TROPIX). The results of the study are shown in FIG. 10.

### EXAMPLE 6

### Secretion of HCV-SEAP in the Serum of Mice Transduced with Adenoviral Vectors Carrying the HCV WT and MT NS3•4A Reporter Constructs

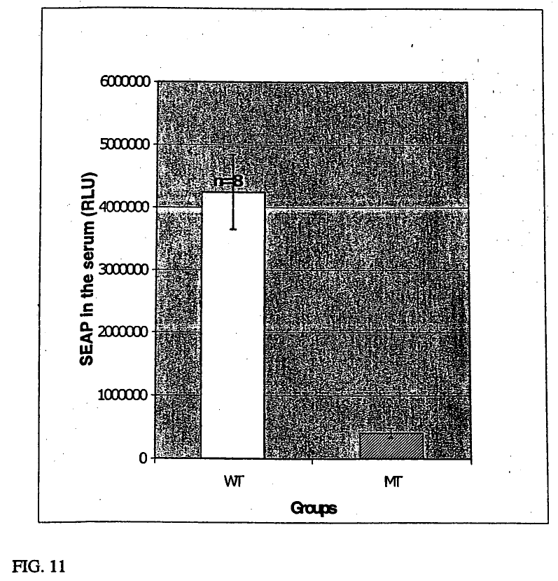

To examine and compare the amount of SEAP secreted in mice transduced with adenovirus containing WT versus MT HCV NS3•4A —SEAP, each set of virus was injected into the tail vein of SCID mice. SEAP was measured in the serum of these mice and the results are shown in FIG. 11. The results are significant at P<0.001.

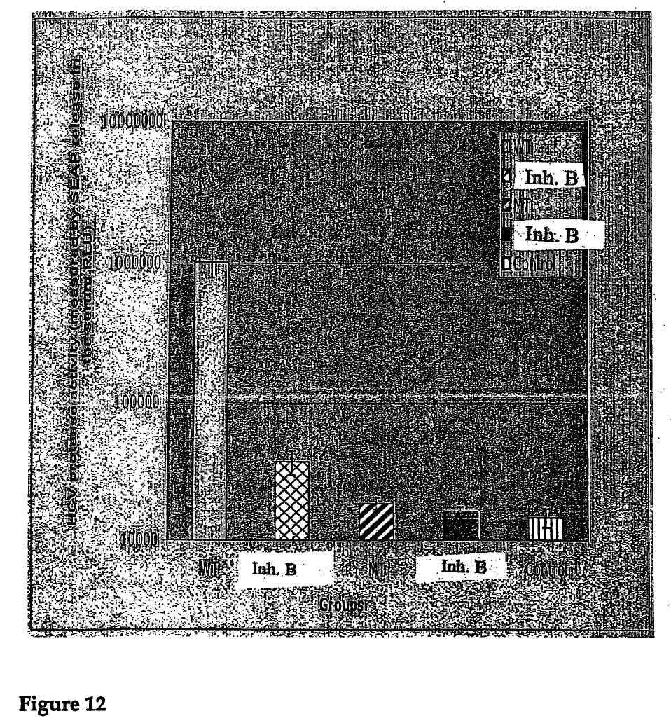

To study whether a HCV protease inhibitor could inhibit the secretion of SEAP under the control of WT HCV protease, but not MT protease, 6 week old SCID mice were injected with 10¹⁰ or 10^{9.5} IFU adenovirus expressing either HCV WT or HCV MT proteins fused to SEAP reporter gene. Mice were dosed with 300 mg/kg of Inhibitor B in Niro suspension vehicle twice a day for two days. After 72 hours mice were bled and the levels of SEAP in the serum were estimated using a BD Biosciences kit after 1:500 dilution of the serum. The results of the study are shown in FIG. 12.

### EXAMPLE 7

### Model For Pathogenesis of HCV Liver Disease

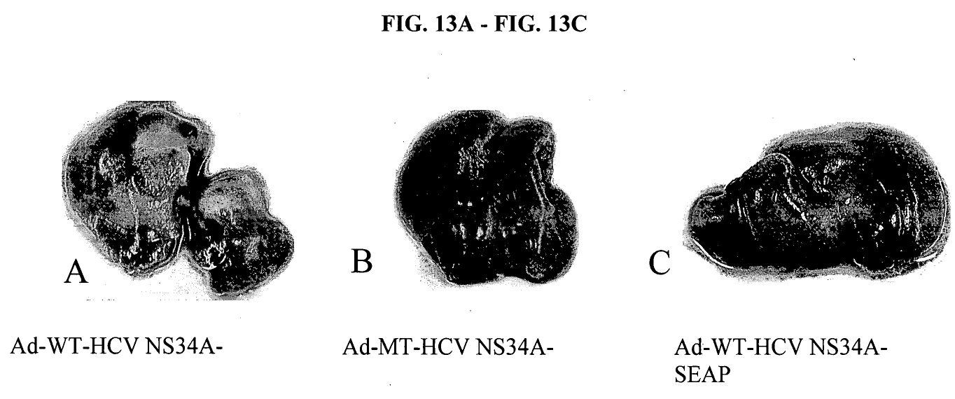

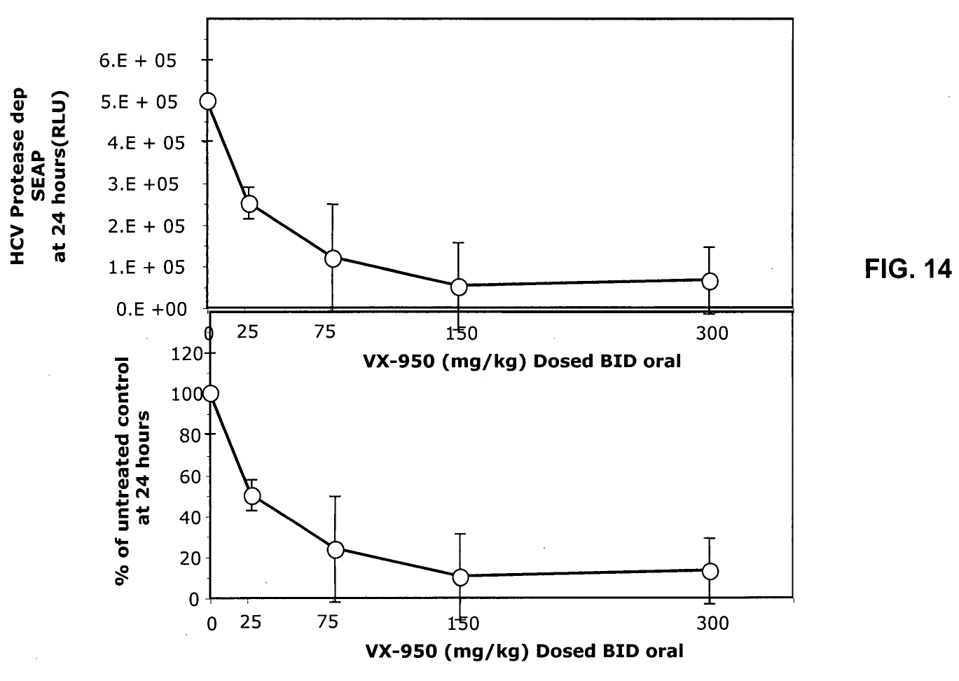

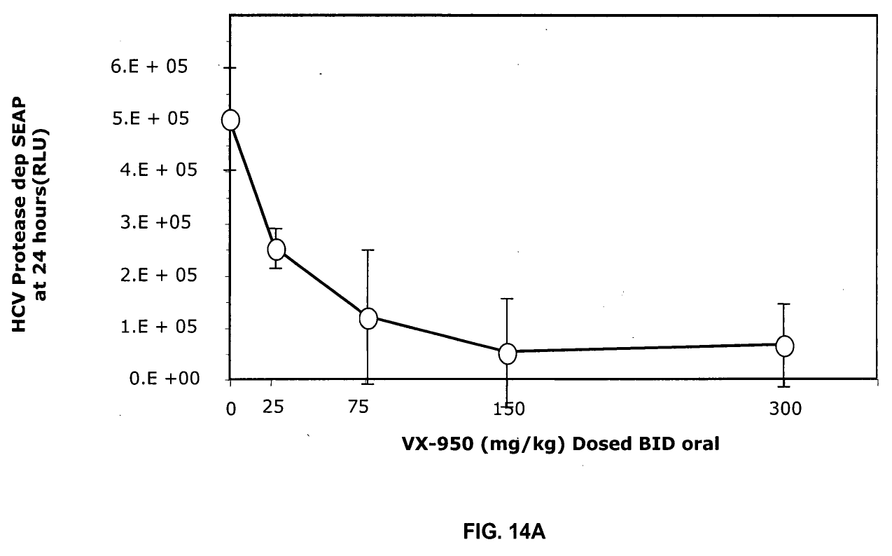

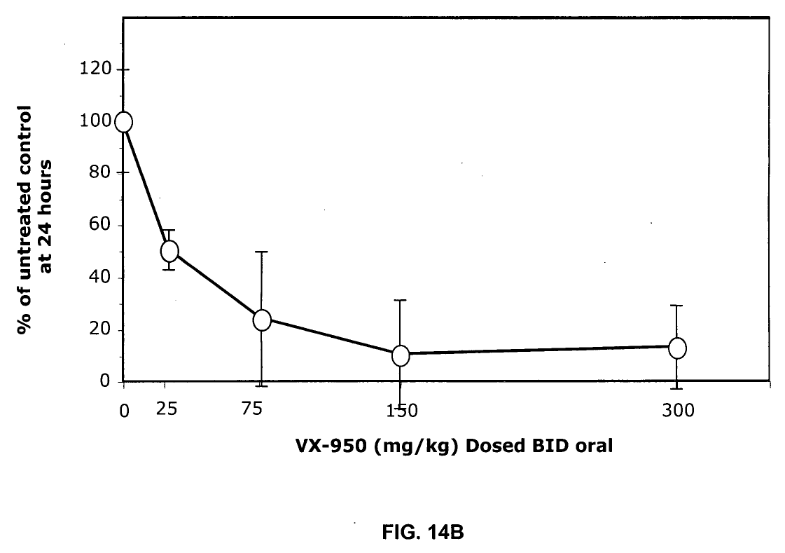

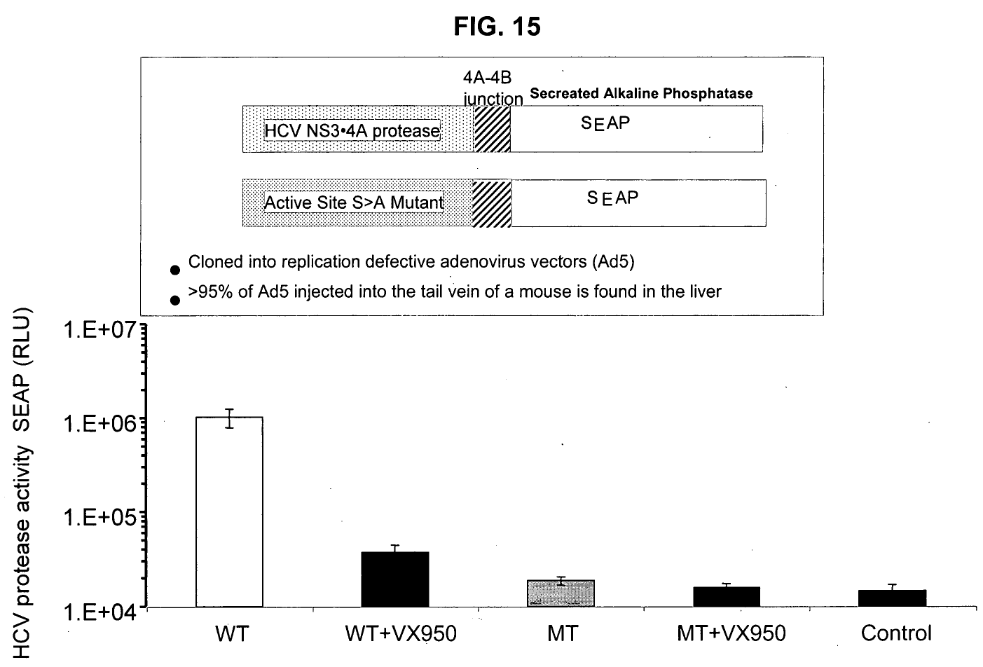

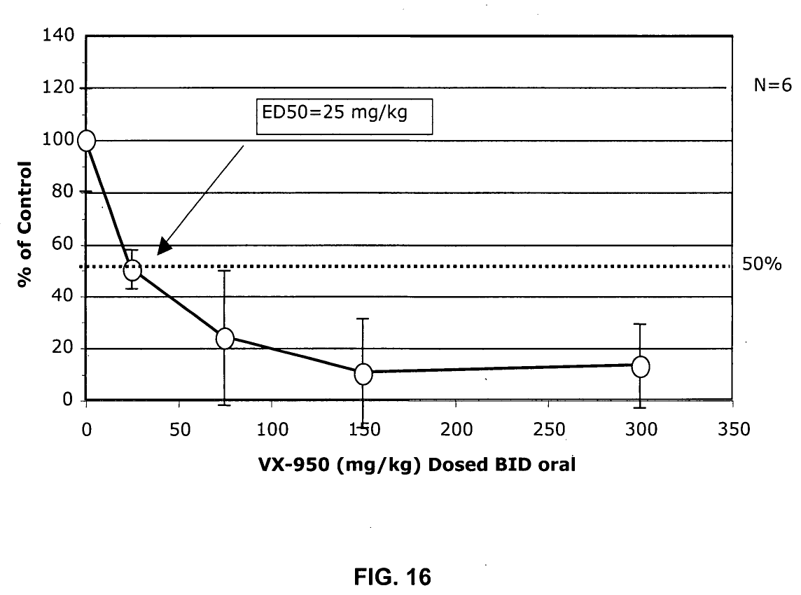

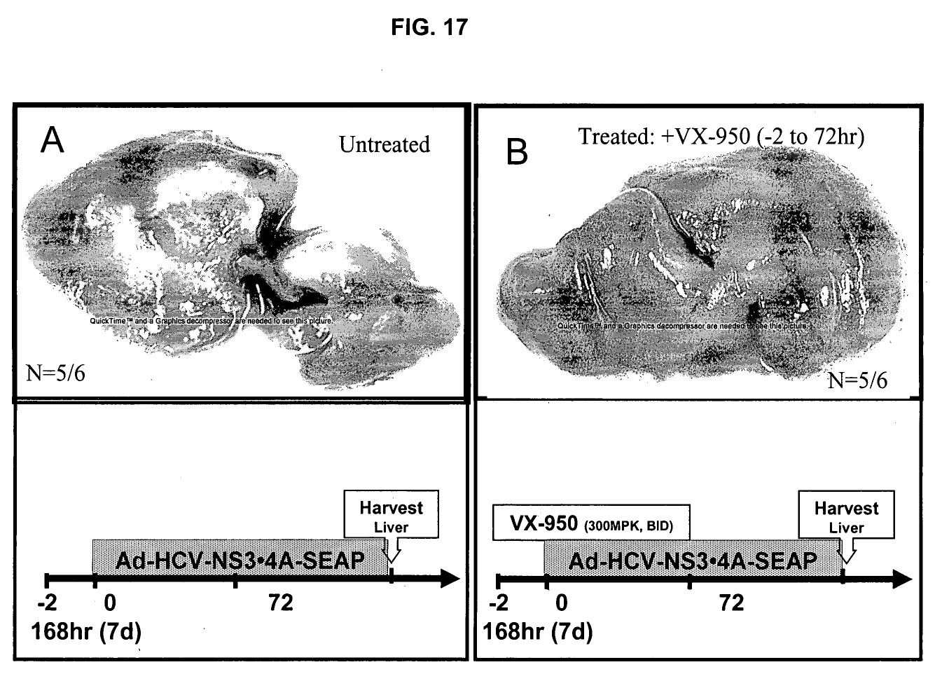

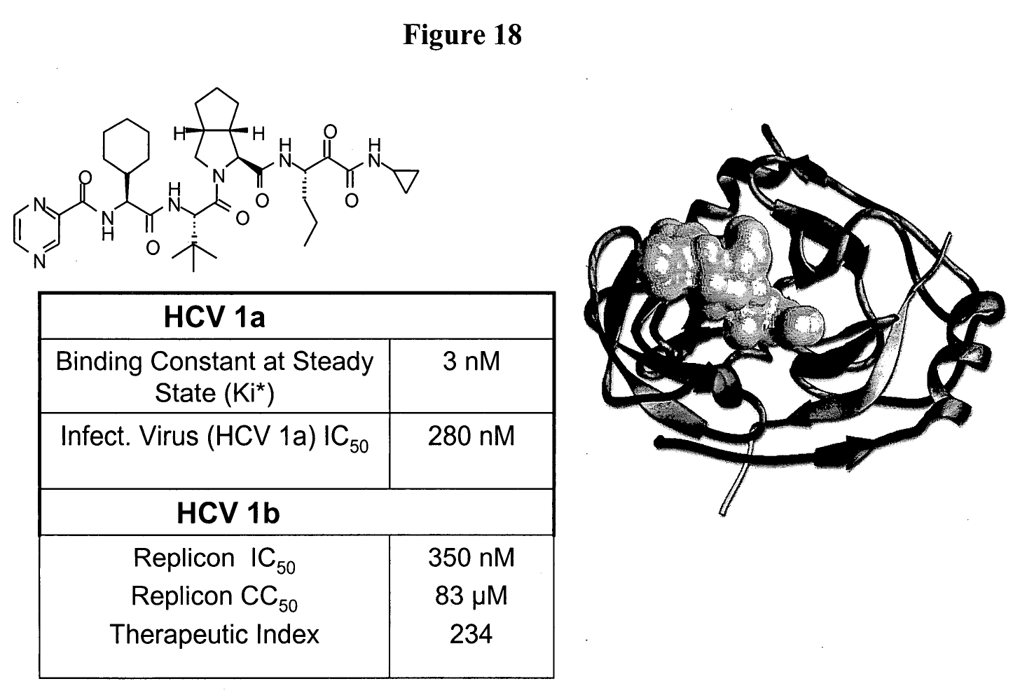

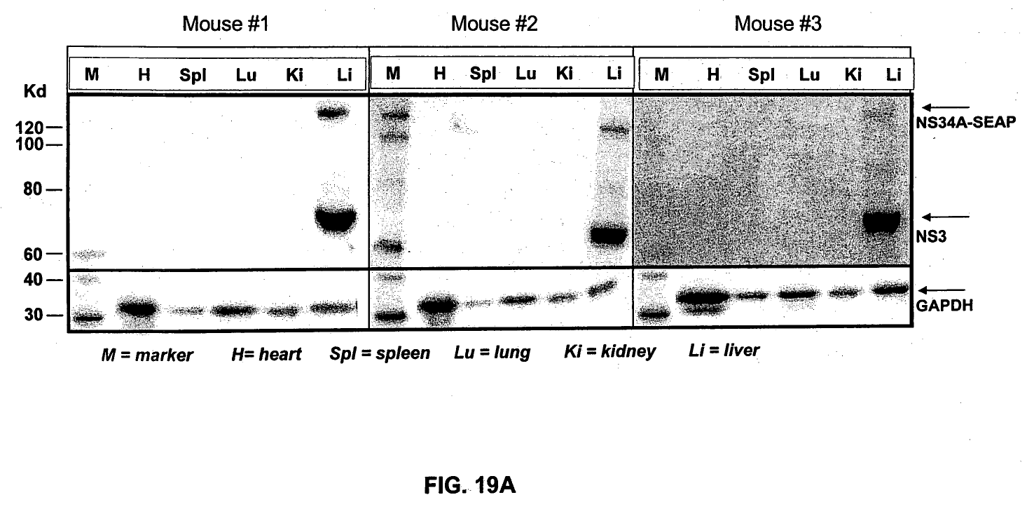

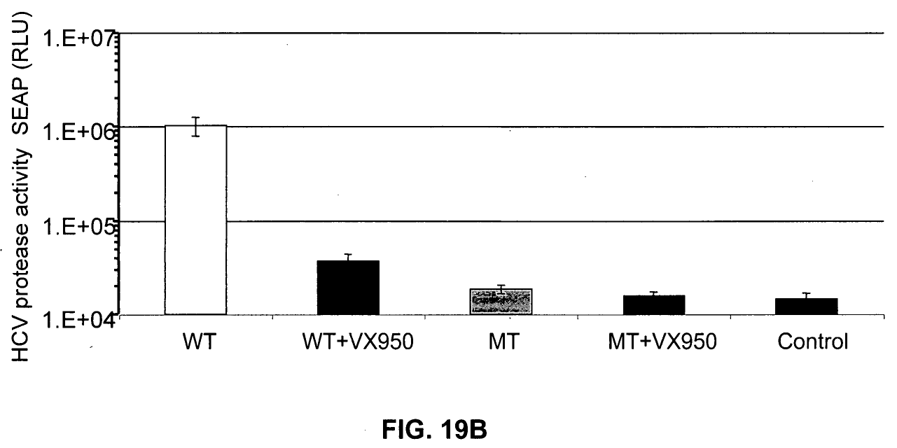

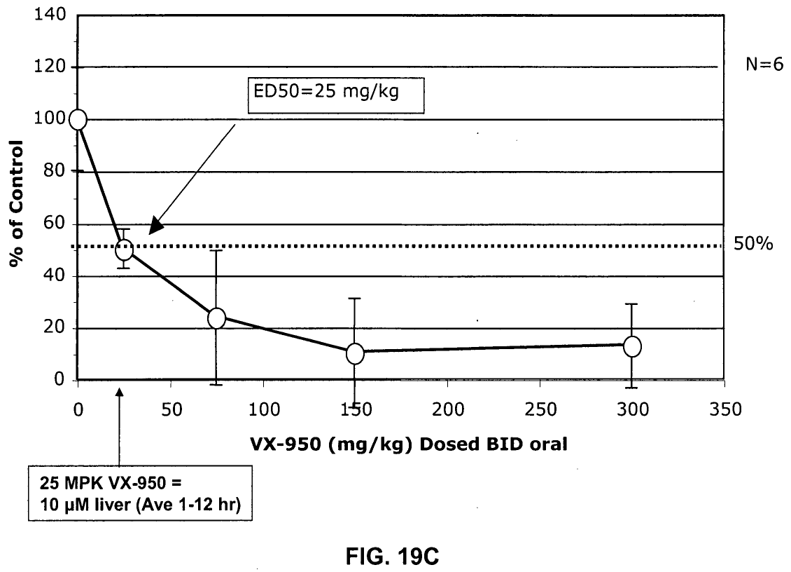

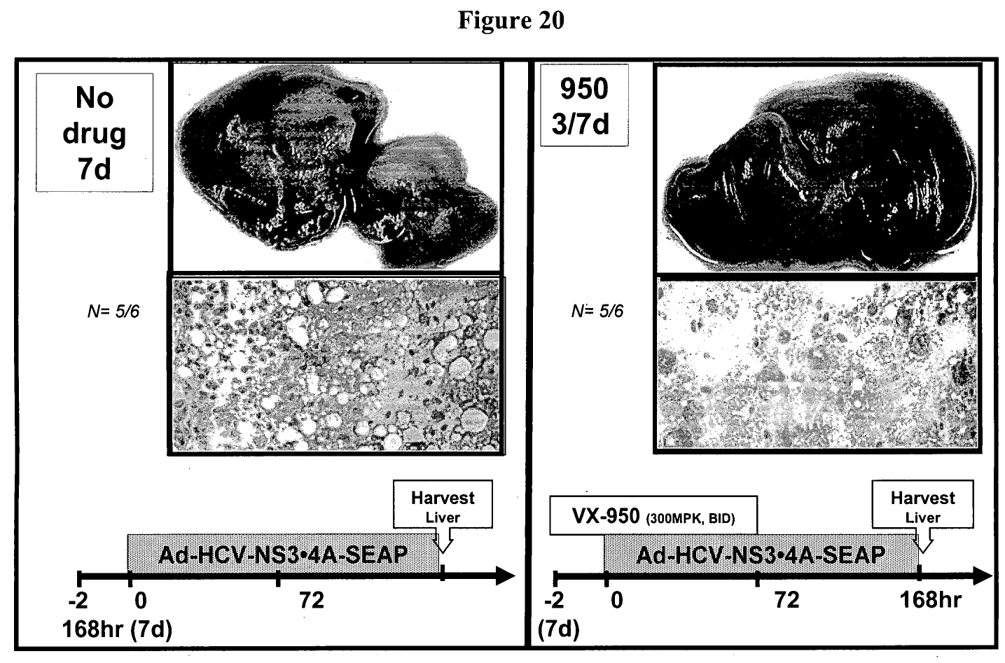

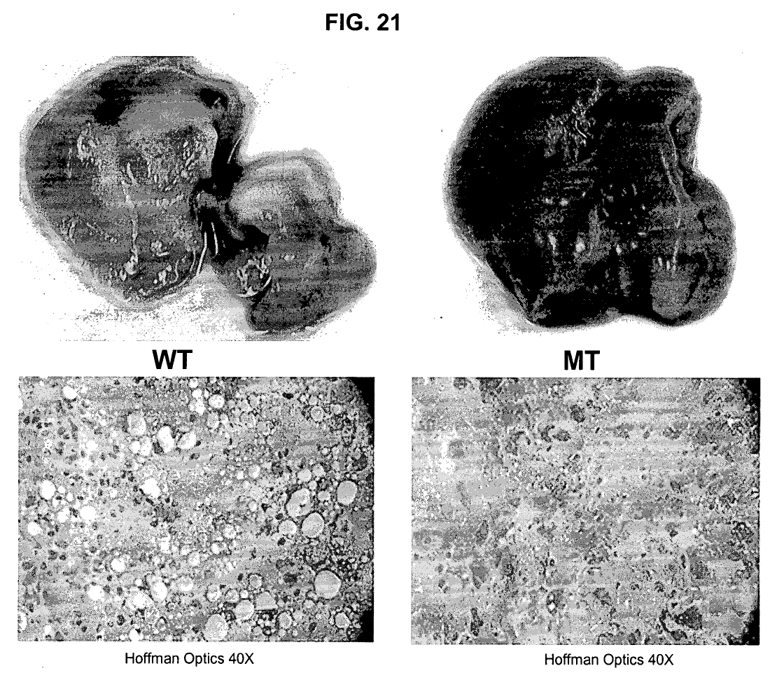

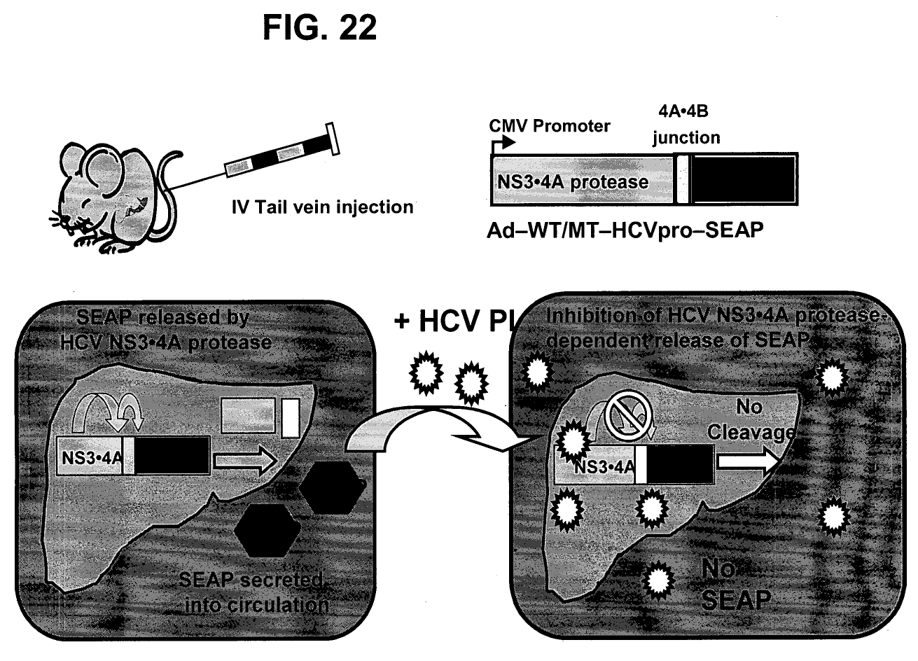

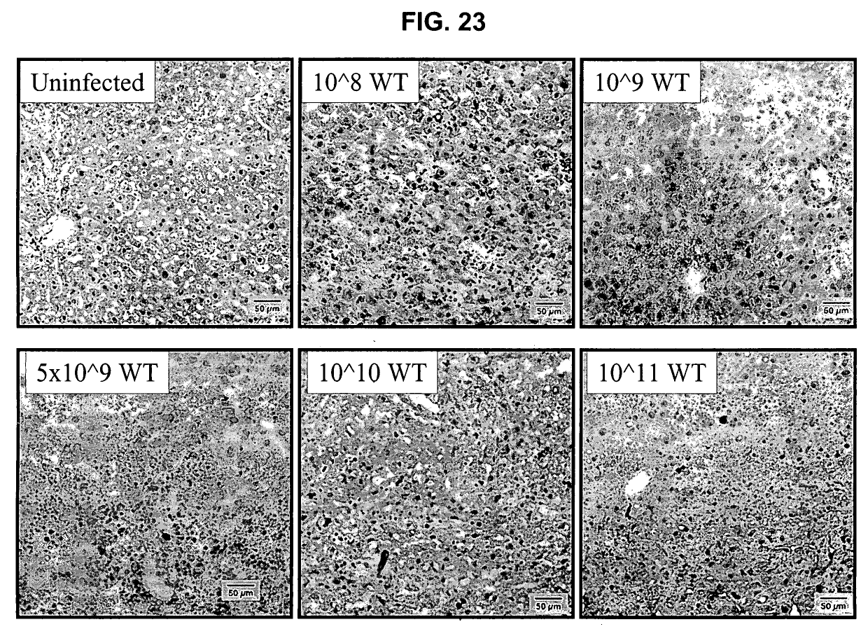

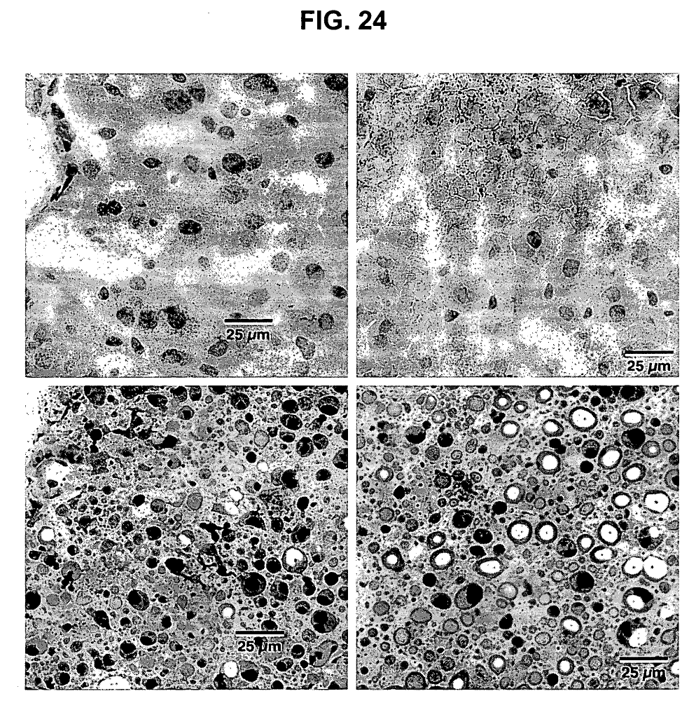

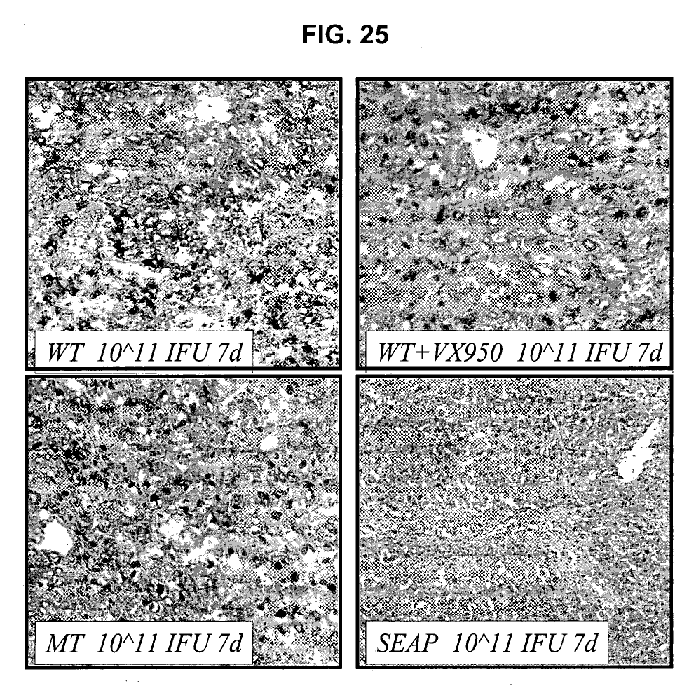

Six week old SCID mice were injected (tail vein injection) with the indicated adenovirus constructs (10¹¹ IFU/mice) expressing either Wild type (WT) or mutant (MT) HCV protease. One group of mice (n=8) injected with the WT virus was treated with HCV protease inhibitor (Inhibitor B, 300 mg/kg BID) for 3 days. Animals were sacrificed after 7 days and the liver were harvested for pathology. Gross morphological changes are shown in FIG. 13.

### EXAMPLE 8

### Oil Red O Staining for Fatty Livers (Steatosis) and Nuclear Counterstaining with Haematoxylin Counter-Stain

1. Cryostat frozen sections of livers stored at −80° C. were tested.

2. The Oil Red O Solution was prepared as follows:

a) Stock Solution Oil Red O: 500 mg Oil red O stain (Sigma O-0625) was dissolved in 100 ml isopropanol at room temperature overnight with stirring. The solution was filtered through a #1 Whatman filter. The solution was stored at room temperature protected from light.

b) Working Oil Red Solution was prepared fresh the day of assay. 40% water was mixed with 60% Oil Red O stock by inversion.

c) The working solution was kept at room temperature for 1 hour before filtering. Particulate material was filtered out using a #1 Whatman filter paper.

3. The slides were fixed in 10% formalin in PBS for 2 hours at room temperature.

4. The fixed slides were carefully rinsed with DI water 3 times for 5 minutes. Care was always taken when rinsing slides to not wash off the liver thin sections.

5. Fixed slides were stained with working Oil Red O solution for 2 hours at room temperature.

6. Fixed and stained slides were carefully rinsed with DI water for 1 hour. A second DI water wash was done overnight and a 3^{rd} for an additional hour.

7. Nuclear counter staining was done with Harris Heamatoxylin for 1 minute.

8. The slides were rinsed once in tap water for 5 minutes.

9. The slides were rinsed with DI water 2 more times for 5 minutes.

10. The slides were mounted in aqueous mounting medium (Biomeda-Gel/mount with anti Fading #MO1D) to prevent extraction of the oil into the medium.

11. The slide edges were sealed with clear nail polish.

12. The slides were stored flat at 6° C.

13. The results were assessed by microscopy.

References

Histopathology Methods 4. (www.hoslink.com/histo/4.HTM) from Flinders Medical centre; Modified Oil Red O Technique, p. 108; Cook H C, Manual of Histological Demonstration Techniques, p 360 Cullings C FA, Handbook of Histopathlogical and Histochemical Techniques 3^{rd} ed; and Histochemistry (1992) 493-497.

### EXAMPLE 9

### Generation of HCV Replicon Cells

Parental Huh-7 cells were cultured in DMEM (Dulbecco's modified Eagle's medium, JRH Biosciences, Lenexa, Kans.), containing 10% heat activated fetal bovine serum (ΔFBS, JRH), supplemented with 2 mM L-glutamine, and nonessential amino acids (JRH). The cells were transfected with an in vitro transcribed sub-genomic HCV replicon RNA identical to the I₃₇₇neo/NS3-3′/wt replicon described by Lohmann et al. (Lohmann, V., F. Korner, J. Koch, U. Herian, L. Theilmann, and R. Bartenschlager. 1999. Replication of subgenomic hepatitis C virus RNAs in a hepatoma cell line. (Science 285:110-3). Stable cells containing the self-replicating HCV replicon were selected and maintained in the presence of 250 μg/ml G418 (Invitrogen, Carlsbad, Calif.), and were then used in the subsequent HCV replicon assays.

### EXAMPLE 10

### Two-Day HCV Replicon Inhibition Assay

HCV replicon cells were plated in a 96-well plate at a density of 10⁴ cells per well in DMEM with 10% ΔFBS to allow the cells to attach and to grow overnight (˜16 h). Then the culture media were removed and replaced with DMEM containing serially diluted compounds (or no compound as a control) in the presence of 2% FBS and 0.5% DMSO. The cells were incubated with the compounds for 48 hours. To determine the antiviral activities of the compounds, total (intracellular) RNA was extracted from the cells using Qiagen RNeasy 96 Kit (Qiagen, Valencia, Calif.). The level of HCV RNA was measured by real-time multiplex quantitative RT-PCR (Taqman, see WO 02/061149) using HCV-specific primers (5′-CCA TGA ATC ACT CCC CTG TG-3′) and 5-CCG GTC GTC CTG GCA ATT C-3′) and a HCV-specific probe (5′-6-FAM-CCT GGA GGC TGC ACG ACA CTC A-TAMRA-3′), and the ABI Prism 7700 Sequence Detection System (Applied Biosystems, Foster City, Calif.). As an internal control for RNA extraction and quantification, a known amount of BVDV RNA was added to each sample prior to extraction and was amplified with specific primers and probe in the multiplexed RT-PCR. Each data point represents the average of five replicates (in cell culture). IC₅₀ is the concentration of the compound at which the HCV RNA level in the replicon cells is reduced by 50%. To monitor cytotoxic effect, the viability of the same replicon cells following 48 h of compound treatment was determined using a tetrazolium compound (MTS)-based assay (CellTiter 96 ® (AQueous One Solution Cell Proliferation Assay, Promega, Madison, Wis.). CC₅₀ is the concentration of the compound at which the cell viability is reduced by 50%.

### EXAMPLE 11

### HCV Ki Assay Protocol

HPLC Microbore Method for Separation of 5AB Substrate and Products Substrate:
NH₂-Glu-Asp-Val-Val-(alpha)Abu-Cys-Ser-Met-Ser-Tyr-COOH

A stock solution of 20 mM 5AB (or concentration of your choice) is made in DMSO w/0.2M DTT. This is stored in aliquots at −20 C.

Buffer: 50 mM HEPES, pH 7.8; 20% glycerol; 100 mM NaCl
Total assay volume was 100 μL

| | X1 | | |
|---|---|---|---|
| | (μL) | conc. in assay | |
| Buffer | 86.5 | See above | |
| 5 mM KK4A | 0.5 | 25 | μM |
| 1 M DTT | 0.5 | 5 | mM |
| DMSO or inhibitor | 2.5 | 2.5% | v/v |
| 50 μM tNS3 | 0.05 | 25 | nM |
| 250 μM 5AB (initiate) | 20 | 25 | μM |

The buffer, KK4A, DTT, and tNS3 are combined; distributed 78 μL each into wells of 96 well plate. This is incubated at 30 C for ˜5-10 min.

2.5 μL of appropriate concentration of test compound is dissolved in DMSO (DMSO only for control) and added to each well. This is incubated at room temperature for 15 min.

Initiated reaction by addition of 20 μL of 250 μM 5AB substrate (25 μM concentration is equivalent or slightly lower than the Km for 5AB).

Incubate for 20 min at 30 C.

Terminate reaction by addition of 25 μL of 10% TFA.

Transfer 120 μL aliquots to HPLC vials.

Separate SMSY product from substrate and KK4A by the following method:

Microbore Separation Method:

Instrumentation: Agilent 1100
Degasser G1322A
Binary pump G1312A
Autosampler G1313A
   Column thermostated chamber G1316A
   Diode array detector G1315A

Column:

Phenomenex Jupiter; 5 micron C18; 300 angstroms; 150×2 mm; P/O 00F-4053-B0

Column thermostat: 40 C.
Injection volume: 100 μL
Solvent A = HPLC grade water + 0.1% TFA
Solvent B = HPLC grade acetonitrile + 0.1% TFA

| | | Flow | |
|---|---|---|---|
| Time (min) | % B | (ml/min) | Max press. |
| 0 | 5 | 0.2 | 400 |
| 12 | 60 | 0.2 | 400 |
| 13 | 100 | 0.2 | 400 |
| 16 | 100 | 0.2 | 400 |
| 17 | 5 | 0.2 | 400 |

Stop time: 17 min
Post-run time: 10 min.

As used herein the term comprising, indicates the potential inclusion of other agents or elements in addition to the specified agents or elements.

All documents cited herein are hereby incorporated by reference.

While we have described a number of embodiments of this invention, it is apparent that our basic examples may be altered to provide other embodiments which utilize the compounds and methods of this invention. Therefore, it will be appreciated that the scope of this invention is to be defined by the appended claims rather than by the specific embodiments that have been represented by way of example above.

1. A non-human mammal whose liver comprises a gene system comprising:
A) a promoter;
B) DNA encoding a protease, and
C) DNA encoding a reporter, wherein a, b, and c are operably linked, and wherein a presence of reporter activity is indicative of protease activity.

2. (canceled)

3. (canceled)

4. (canceled)

5. A non-human mammal whose liver comprises a gene system comprising an operably linked promoter and DNA encoding a protein whose expression causes liver damage.

6-16. (canceled)

17. Cells obtained from the non-human animal according to claim 5, wherein the cells comprise said gene system.

18. A hepatocyte or a hepatocyte cell line comprising a gene system comprising A) a promoter; B) DNA encoding a protease, and C) DNA encoding a reporter, wherein A, B, and C are operably linked, and wherein a presence of reporter activity is indicative of protease activity.

19. A hepatocyte or a hepatocyte cell line comprising a promoter and DNA encoding a protein whose expression causes hepatocyte damage.

20. A viral vector comprising A) a promoter; B) DNA encoding a protease; and C) DNA encoding a reporter, wherein A, B, and C are operably linked.

21. A viral vector comprising an operably linked a promoter and DNA encoding a protein whose expression causes liver damage.

22. A process for producing a mammalian model for protease activity, the method comprising:
providing an mammal; and
delivering to the mammal a gene system comprising: A) a promoter; B) DNA encoding a proteas; and C) DNA encoding a reporter, wherein A, B, and C are operably linked, and wherein a presence of reporter activity is indicative of protease activity.

23. (canceled)

24. (canceled)

25. (canceled)

26. A process for producing a mammalian model for liver damage, the method comprising:
a) providing a mammal; and
b) delivering to the mammal a gene system comprising a promoter and DNA encoding a protein whose expression causes hepatocyte damage.

27-37. (canceled)

38. A non-transgenic, non-human mammal wherein the liver cells of said mammal comprise an expression construct comprising:
A) a promoter;
B) DNA encoding a protease; and
C) DNA encoding a reporter, wherein A, B, and C are operably linked, and wherein expression of said construct is detected by the presence of reporter activity and is indicative of protease activity.

39. (canceled)

40. (canceled)

41. (canceled)

42. A non-human mammal whose liver comprises an expression construct comprising an operably linked promoter and DNA encoding a protein whose expression causes liver damage.

43-53. (canceled)

54. Cells obtained from the non-human animal according to claim 38, wherein the cells express the reporter gene from said expression construct.

55. A hepatocyte or a hepatocyte cell line transformed or transfected with an expression construct that comprises A) a promoter; B) DNA encoding a protease; and C) DNA encoding a reporter, wherein A, B, and C are operably linked, and wherein said hepatocyte or hepatocyte cell line expresses a reporter activity.

56. A method of producing a non-transgenic, non-human mammalian model for protease activity, the method comprising the steps of administering to a non-human mammal a composition comprising an expression construct that comprises A) a promoter; B) DNA encoding a protease; and C) DNA encoding a reporter, wherein A, B, and C are operably linked, in an amount effective produce the expression of said protease in the cells of said animal, wherein a presence of reporter activity is indicative of expression of said protease in said animal.

57. (canceled)

58. (canceled)

59. (canceled)

60. A process for producing a non-transgenic non-human mammalian model for liver damage, comprising administering to a non-human mammal a composition comprising an expression construct comprising a promoter and DNA encoding a protein whose expression causes hepatocyte damage.

61-71. (canceled)

72. A method for testing an agent which augments or inhibits protease activity, the method comprising:
a) providing a mammal according to claim 1;
b) administering the agent to the mammal; and
c) evaluating the effect of the agent on the reporter expression.

73. A method for assessing an agent which augments or inhibits liver damage, comprising:
a) providing a mammal according to claim 5;
b) administering the agent to the mammal; and
c) evaluating the effect of the agent on the damage.

74. A method for identifying a compound that modulates steatosis comprising:
a) providing a mammal according to claim 5;
b) administering a compound to the mammal; and
c) evaluating the effect of the compound on steatosis in the mammal.

75. A method for identifying a compound for treating NAFLD, NASH, alcoholic steatosis, or Reye's syndrome comprising:
a) providing a mammal according to claim 5;
b) administering a compound to the mammal; and
c) selecting the compound that treats or ameliorates the effects of NAFLD, NASH, alcoholic steatosis, or Reye's syndrome.

76. A method for treating steatosis or fatty liver in a mammal comprising administering to the mammal a HCV NS3•4A protease inhibitor.

77. A method for hepatoprotection in a mammal comprising administering to the mammal a HCV NS3•4A protease inhibitor.

78. A method for treating NAFLD, NASH, alcoholic steatosis, or Reye's syndrome in a mammal comprising administering to the mammal a HCV NS3•4A protease inhibitor.

79. The method according to claim 75 wherein the protease inhibitor is VX-950.

80. The method according to claim 76 wherein the protease inhibitor is VX-950

81. The method according to claim 77 wherein the protease inhibitor is VX-950
